(19) United States
(12) Patent Application Publication
(10) Pub. No.: US 2005/0238651 A1
(43) Pub. Date: Oct. 27, 2005

(54) Treatment of inflammatory bowel disease

(76) Inventors: Gregory J. Gurtner, Ridgefield, CT (US); William F. Stenson, Ladue, MO (US); Nancy Gurtner, Ridgefield, CT (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/095,946

(22) Filed: Mar. 31, 2005

Related U.S. Application Data
(63) Continuation-in-part of application No. 10/997,147, filed on Nov. 24, 2004.
(60) Provisional application No. 60/531,587, filed on Dec. 19, 2003.
(60) Provisional application No. 60/524,753, filed on Nov. 25, 2003.

(51) Int. Cl. A61K 45/00; A61K 39/00

(52) U.S. Cl. 424/184.1; 424/93.7; 514/166; 514/171

(57) ABSTRACT

Methods and compositions are disclosed for treatment of inflammatory bowel disease in a patient in need thereof based upon administration of an inducer of indoleamine 2,3-dioxygenase, a ligand of B7 antigen expressed on antigen presenting cells, or a combination thereof.

### CROSS-REFERENCE TO RELATED APPLICATIONS

The present application is a continuation-in-part of U.S. patent application No. 10/997,147 filed Nov. 24, 2004, which claims priority to U.S. Provisional Application No. 60/531,587, filed Dec. 19, 2003; and U.S. Provisional Application No. 60/524,753, filed Nov. 25, 2003. These applications are incorporated herein in their entireties by reference.

### STATEMENT REGARDING FEDERALLY SPONSORED RESEARCH OR DEVELOPMENT

This work was supported at least in part with funds from the federal government under U.S.P.H.S. Grant P30 DK52574 awarded by the National Institutes of Health. The U.S. Government may have certain rights in the work presented herein.

### FIELD

This application relates generally to Inflammatory Bowel Diseases and, more particularly, to methods and compositions for treating Inflammatory Bowel Diseases.

### BACKGROUND

Inflammatory bowel diseases including Crohn's disease and ulcerative colitis, are chronic inflammatory disorders of the gastrointestinal tract resulting from upregulation of the mucosal immune system. Current treatment approaches involve the use of anti-inflammatory agents, aminosalicylates and corticosteroids. (for review, see Hibi et al., Journal of Gastroenterology 38 Suppl. 15, 36-42, 2003). Nevertheless, these therapies do not successfully treat all patients, and in patients in whom the therapies are effective, unpleasant side effects are often seen (Sawada, Diseases of the Colon & Rectum 46(10 Suppl), S66-S77, 2003). Thus, there remains a need for new therapeutic approaches.

### SUMMARY

Accordingly, the present inventors have succeeded in discovering that increased expression of indoleamine 2,3-dioxygenase in antigen presenting cells of the gastrointestinal tract produces a downregulation of the proliferative response of Th1 helper-T cells during inflammation. As a result, substances that increase concentration or activity of indoleamine 2,3-dioxygenase in antigen presenting cells of the gastrointestinal tract, decrease the inflammatory response in patients having inflammatory bowel disease.

Thus, in various embodiments, the present invention involves methods for treating a patient having inflammatory bowel disease. In certain configurations, the method comprises administering to the patient an anti-inflammatory amount of an inducer of indoleamine 2,3-dioxygenase in antigen presenting cells of the patient's gastrointestinal tract. Such increase in enzyme activity reduces inflammation in the gastrointestinal tract and thereby provides a new approach for treating inflammatory bowel disease.

In various of the embodiments of the present invention the inflammatory bowel disease can be ulcerative colitis or Crohn's disease and the substance administered can increase expression of indoleamine 2,3-dioxygenase in the antigen presenting cells. Non-limiting examples of inducers of indoleamine 2,3-dioxygenase include a bacterial lipopolysaccharide, a cytokine such as interferon-gamma or a cytotoxic T lymphocyte-associated antigen. The cytokine or cytotoxic T lymphocyte-associated antigen 4 can be in the form of a fusion polypeptide or a pegylated polypeptide.

The present inventors have also succeeded in discovering that administering a ligand of a B7 antigen displayed on antigen presenting cells of a gastrointestinal tract of a patient suffering from inflammatory bowel disease such as Crohn's disease or ulcerative colitis, can ameliorate or abrogate the symptoms of inflammatory bowel disease. The antigen presenting cells comprised by the gastrointestinal tract of a patient can be antigen presenting cells comprised by the colon of the patient. B7 antigens are cell-surface antigens comprised by antigen presenting cells (Finger et al., Nature Immunology 3, 1056-1057, 2002). The inventors have found that binding B7 antigen comprised by colonic antigen presenting cells with certain B7 ligands can promote immune tolerance. Binding of a B7 cell-surface molecule with a ligand can thus result in a costimulatory blockade of T cell activation, which would otherwise be signaled through a CD28 receptor (Finger et al., Nature Immunology 3, 1056-1057, 2002). In addition, the inventors have succeeded in discovering that increased expression of indoleamine 2,3-dioxygenase (IDO) in antigen presenting cells of the gastrointestinal tract produces a downregulation of the proliferative response of Th1 helper-T cells during inflammation. As a result, substances that increase concentration or activity of indoleamine 2,3-dioxygenase in antigen presenting cells of the gastrointestinal tract, can decrease the inflammatory response in patients having inflammatory bowel disease. The inventors have further discovered that administration of ligands of B7which promote tolerance by a costimulatory blockade of T cell activation by antigen presenting cells of the gastrointestinal tract can also lead to an increase in concentration or activity of indoleamine 2,3-dioxygenase in the antigen presenting cells of the gastrointestinal tract.

Accordingly, as a result of their discovery of dual mechanisms for abrogation of inflammatory bowel disease by administration of a B7 ligand, the inventors have developed new approaches for the treatment of inflammatory bowel disease in a patient in need thereof.

Accordingly, the present teachings provide methods of treating inflammatory bowel disease in a patient. The methods comprise administering to a patient in need thereof an immune tolerance-promoting amount of a ligand of a B7 antigen comprised by antigen presenting cells of the patient's gastrointestinal tract. In various configurations, contact between a ligand of B7 antigen and antigen presenting cells, which can lead to binding of the ligand of B7 antigen to a B7antigen, can provide a costimulatory blockade to T cell activation by the antigen presenting cells of the patient's gastrointestinal tract. In addition, in various aspects, a ligand of B7 antigen can also induce increased expression of indoleamine 2,3-dioxygenase in antigen presenting cells of the patient's gastrointestinal tract.

In related aspects, administering a ligand of a B7 antigen to a patient for treatment of inflammatory bowel disease can comprise administering the B7 antigen ligand systemically, which can include systemic administration by intravenous infusion. In some embodiments, administering a B7 ligand to a patient in need of treatment for inflammatory bowel disease can further comprise administering at least one substance selected from the group consisting of 5-aminosalicylates, corticosteroids, azathioprine and antibodies directed against tumor necrosis factor-α, such as monoclonal antibody cA2 (infliximab) (Elliott, M. J., et al., Lancet 344, 1105-1110, 1994; Hanauer, S. B., et al., Clinical Therapeutics 20, 1009-1028, 1998).

In various embodiments of the present teachings, the ligand of B7antigen can comprise a cytotoxic T lymphocyte-associated antigen 4 (CTLA-4) (Finck, G. K., et al., Science 265, 1225-1227, 1994; Grohmann, U. et al. Nature Immunology 3, 1097-1101, 2002). In various configurations, the CTLA-4 can be a fusion polypeptide comprising the extracellular domain of a CTLA-4 fused to an Fc portion of an immunoglobulin (CTLA-4-Ig, Finck, G. K., et al., Science 265, 1225-1227, 1994). In various aspects, the CTLA-4 fusion polypeptide can be pegylated (U.S. Pat. No. 4,179,337 to Davis; Francis et al., International Journal Hematology 68, 1-18, 1998).

Various embodiments of the present teachings include methods of downregulating a T helper 1 cell proliferation response in inflammation within the gastrointestinal tract in a mammalian subject having inflammatory bowel disease. These methods can comprise administering to a subject in need of treatment of inflammatory bowel disease a pharmaceutical composition comprising an inducer of indoleamine 2,3-dioxygenase in antigen presenting cells, an immune tolerance-promoting amount of a ligand of a B7 antigen, or both. In some configurations, the inducer of indoleamine 2,3-dioxygenase can increase expression of the enzyme in antigen presenting cells. In certain configurations, the B7 antigen can be comprised by antigen presenting cells of the subject's gastrointestinal tract. In certain aspects, an immune tolerance-promoting amount of a ligand of a B7 antigen can comprise an effective amount of an inducer of indoleamine 2,3-dioxygenase. In some configurations, the inducer can increase expression of indoleamine 2,3 dioxygenase in antigen presenting cells, such as antigen presenting cells of a patient's gastrointestinal tract. Accordingly, in various configurations, an immune tolerance-promoting amount of a ligand of a B7 antigen can also be an inducing amount of an inducer of indoleamine 2,3-dioxygenase. Such amounts can be, in various aspects, amounts of a ligand of B7 clinically effective for abrogating a inflammatory bowel disease such as ulcerative colitis or Crohn's disease. In various configurations, a ligand of B7 which can also be an inducer of indoleamine 2,3-dioxygenase can be a cytotoxic T lymphocyte-associated antigen 4, such as a cytotoxic T lymphocyte-associated antigen 4-immunoglobulin fusion polypeptide described supra.

In various other embodiments, the present teachings are also directed to packaged pharmaceuticals. The packaged pharmaceutical can comprise an anti-inflammatory amount of an inducer of indoleamine 2,3-dioxygenase in antigen presenting cells of a patient having inflammatory bowel disease, and anti-inflammatory amount of a ligand of B7 antigen comprised by antigen presenting cells of a patient's gastrointestinal tract, or both, in a pharmaceutically acceptable formulation. In various configurations, the ligand of B7 antigen can also be the inducer of indoleamine 2,3-dioxygenase. The packaged pharmaceutical can, in various aspects, also include instructions for using the ligand of B7 for treating inflammatory bowel disease in a patient in need thereof.

In various configurations of the present teachings, the antigen presenting cells can be professional antigen presenting cells. Furthermore, the antigen presenting cells can be lamina propria mononuclear cells, macrophages, dendritic cells, or a combination thereof. In some aspects, the patient or subject can be a human patient or subject.

Some embodiments of the present teachings disclose methods of treating inflammatory bowel disease which comprise selecting an agent on the basis of the agent being effective in inducing indoleamine 2,3-dioxygenase in antigen presenting cells, effective in blockading costimulation of T cell activation, or effective in both inducing indoleamine 2,3-dioxygenase in antigen presenting cells and blockading costimulation of T cell activation, and administering an effect amount of the agent to a patient in need thereof.

In certain embodiments, if the agent is selected on the basis of being effective in inducing indoleamine 2,3-dioxygenase in antigen presenting cells, the antigen presenting cells can be lamina propria mononuclear cells, macrophages, dendritic cells or a combination thereof. In some aspects, the agent can be a bacterial lipopolysaccharide, an interferon-y, or a cytotoxic T lymphocyte-associated antigen 4, such as a cytotoxic T lymphocyte-associated antigen 4-Ig fusion polypeptide, a pegylated cytotoxic T lymphocyte-associated protein 4-Ig, or a combination thereof.

In certain embodiments, if the agent is selected on the basis of being effective in blockading costimulation of T cell activation, the agent can be a cytotoxic T lymphocyte-associated antigen 4, such as a cytotoxic T lymphocyte-associated antigen 4-Ig fusion polypeptide, a pegylated cytotoxic T lymphocyte-associated protein 4-Ig or a combination thereof. Furthermore, in various configurations, the antigen presenting cells can be, for example, professional antigen presenting cells such as lamina propria mononuclear cells, macrophages, dendritic cells or a combination thereof.

In various configurations, a method of treating inflammatory bowel disease can further comprise monitoring a patient receiving treatment for inflammatory bowel disease as disclosed herein. In various aspects, the monitoring can comprise monitoring the patient for a response to an indoleamine 2,3-dioxygenase-inducing agent, or a response to a T-cell costimulation blockading agent, wherein the response can be indicative of therapeutic benefit for treatment of inflammatory bowel disease. The monitoring can comprise any method of monitoring progression of inflammatory bowel disease known to skilled artisans, for example methods presented in references such as Miehsler, W., et al., Inflammatory Bowel Diseases 7, 99-105, 2001; Aberra, F. N., et al., Alimentary Pharmacology & Therapeutics 21, 307-319, 2005; Rizzello, R., et al., Alimentary Pharmacology & Therapeutics 16 Suppl. 4, 3-6, 2002; Cunliffe, R. N. et al., Alimentary Pharmacology & Therapeutics 16, 647-662, 2002; and Sostegni, R., et al., Alimentary Pharmacology & Therapeutics 17 Suppl. 2, 11-17, 2003. Methods of monitoring can include, in certain alternative aspects, detecting an increase in indoleamine 2,3-dioxygenase expression in the antigen presenting cells of the patient's gastrointestinal tract. Such an increase in expression can be, for example, an increase in the levels of indoleamine 2,3-dioxygenase protein or the levels of indoleamine 2,3-dioxygenase mRNA in the antigen presenting cells of the patient's gastrointestinal tract following administration of an agent effective in inducing 2,3 dioxygenase (compared to levels prior to administration of the agent). Methods of monitoring can also comprise monitoring a blockade of costimulation of T cell activation. Monitoring of a costimulatory blockade of T cell activation can comprise, in non-limiting example, monitoring a downregulation of T helper 1 cell proliferation following administration of an agent that promotes T cell costimulation blockade. T helper 1 cell proliferation can be monitored by methods known to skilled artisans, such as, in non-limiting example, flow cytometry.

### BRIEF DESCRIPTION OF THE DRAWINGS

FIG. 1 illustrates increased Indoleamine 2,3-dioxygenase expression in the distal colon in Trinitrobenzene Sulfonic Acid colitis as demonstrated by A) Real-Time PCR and B) Western blotting.

FIG. 2 illustrates increased Indoleamine 2,3-dioxygenase expression in lamina propria mononuclear cells in Trinitrobenzene Sulfonic Acid-treated colons and decreased quinolinic acid expression in Trinitrobenzene Sulfonic Acid-treated colons treated with 1-methyl tryptophan (1-mT).

FIG. 3 illustrates worsening colitis and increased mortality in Trinitrobenzene Sulfonic Acid colitis with Indoleamine 2,3-dioxygenase inhibition in a mouse model of inflammatory bowel disease.

FIG. 4 illustrates that Indoleamine 2,3-dioxygenase inhibition leads to toxic colonic dilation in the setting of Trinitrobenzene Sulfonic Acid colitis.

FIG. 5 illustrates the increase in cytokines expressed in colons of mice treated with Trinitrobenzene Sulfonic Acid and placebo or 1-methyl tryptophan using Real-Time PCR to quantify mRNA levels.

FIG. 6 illustrates Indoleamine 2,3-dioxygenase expression in lamina propria mononuclear cell subpopulations isolated by fractionation using magnetic selection columns.

FIG. 7 illustrates decreased Indoleamine 2,3-dioxygenase expression and increased inflammation in STAT-1 knockout mice exposed to Trinitrobenzene Sulfonic Acid.

FIG. 8 illustrates decreased Indoleamine 2,3-dioxygenase expression in IFN-γ Receptor and STAT-1 knockout mice.

FIG. 9 illustrates that inhibition of Indoleamine 2,3-dioxygenase by 1-methyl tryptophan leads to increased lymphocyte proliferation in Trinitrobenzene Sulfonic Acid colitis as determined by BrdU immunohistochemistry.

FIG. 10 illustrates that LPS or CTLA-4-Ig administration induces Indoleamine 2,3-dioxygenase expression in lamina propria mononuclear cells and LPS downregulates the inflammatory response to Trinitrobenzene Sulfonic Acid.

FIG. 11 illustrates weight loss in CTLA-4-Ig and Trinitrobenzene Sulfonic Acid-treated mice.

FIG. 12 illustrates survival in CTLA-4-Ig and Trinitrobenzene Sulfonic Acid treated mice.

FIG. 13 illustrates clinical characteristics of CTLA-4-Ig and TNBS treated mice.

FIG. 14 illustrates histology of CTLA-4-Ig and Trinitrobenzene Sulfonic Acid treated mice.

FIG. 15 illustrates histological and morphological scoring in CTLA-4-Ig and TNBS treated mice.

FIG. 16 illustrates CTLA-4-Ig induction of IDO in cultured colonic lamina propria mononuclear cells

FIG. 17 illustrates CTLA-4-Ig Induction of Colonic IDO and IFN-γ expression.

FIG. 18 illustrates colonic indoleamine 2,3-dioxygenase protein expression increasing in response to CTLA-4-Ig in TNBS colitis

FIG. 19 illustrates that CTLA-4-Ig can mediate inhibition of colonic TNFα mRNA expression without affecting IL12 mRNA expression in the setting of active TNBS colitis.

FIG. 20 illustrates that CTLA-4-Ig can mediate colonic TGFβ1 expression without altering Foxp3 expression.

### DETAILED DESCRIPTION

The methods and compositions described herein utilize laboratory techniques well known to skilled artisans and can be found in laboratory manuals such as Sambrook, J., et al., Molecular Cloning: A Laboratory Manual, 3 rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999.

The inventors herein have discovered that interference with the ability of T cell CD28 to interact with a B7 molecule on a professional antigen presenting cell through the binding of a B7 ligand to a B7 molecule on the surface of an antigen presenting cell can promote immune tolerance, and thereby alleviate symptoms of inflammatory bowel disease. As used herein, immune tolerance describes a state of unresponsiveness by the immune system of an individual to one or more antigens to which the individual is expected to be responsive. Immune tolerance as used herein can include a reduction or lack of activation of T cells due to interference with antigen presenting cell-T cell interactions.

The inventors have further discovered that the inflammation response in inflammatory bowel disease can be ameliorated by administering to a patient in need thereof an agent which acts as an inducer of indoleamine 2,3-dioxygenase (IDO) (i.e., induces or increases expression of IDO, or transcription of the gene for IDO) in antigen presenting cells of the gastrointestinal tract, by administering an immune tolerance-promoting amount of a ligand of B7 comprised by antigen presenting cells of the patient's gastrointestinal tract, or by a combination thereof. In various configurations, administering the IDO inducer IDO, the ligand of B7 antigen, or a combination thereof can lead to downregulation of the proliferative response of Th1 helper-T cells. In various configurations, the administering a ligand of B7 antigen can also provide a costimulatory blockade of T cell activation by antigen presenting cells of the gastrointestinal tract, and can act as an inducer of indoleamine 2,3-dioxygenase in antigen presenting cells of the gastrointestinal tract. As used herein, the term “inducer” or “inducers” refers to compounds that increase activity of indoleamine 2,3-dioxygenase in a cell by increasing the amount of the enzyme accumulated the cell or by increasing the substrate turnover rate. A “costimulatory blockade,” as used herein, refers to an interference with the ability of T cell CD28 to interact with a B7 molecule on a professional antigen presenting cell, such as an antigen presenting cell comprised by a patient's gastrointestinal tract. As used herein, the term “professional antigen presenting cells” refers to cells whose predominant functions include presentation of antigens to T cells (Lassila, O., et al., Nature 334, 253-255, 1988). Non-limiting examples of professional antigen presenting cells include macrophages and dendritic cells.

Antigen presenting cells of the present teachings can include, in various embodiments, cells which can present antigens to T cells, and can include antigen presenting cells located in the body in, on, around, or adjacent to the body's gastrointestinal tract. The antigen presenting cells of the present teachings can comprise, in non-limiting example, lamina propria mononuclear cells, macrophages, and dendritic cells.

Diagnosis of inflammatory bowel disease can be based upon evaluation of a patient's clinical, radiographic, endoscopic, and histopathologic features. (for review see Papadakis et al., Gastrointestinal Endoscopy Clinics of North America 12, 433-449, 2002; Chutkan et al., Gastrointestinal Endoscopy Clinics of North America 12, 463-483, 2002; Fishman, Canadian Journal of Gastroenterology. 15, 627-628, 2001). The inventors herein have shown that expression of indoleamine 2,3-dioxygenase is increased in an animal model of colitis predictive of inflammatory bowel disease in humans (for review see Neurath et al., International Review of Immunology 19, 51-62, 2000), and that inhibition of the IDO enzyme leads to a worsening of colitis symptoms in the animal model, including increased mortality. Hence, detection of this increase in expression can be of benefit in the diagnosis of inflammatory bowel disease, i.e. in distinguishing the disease from other diseases. Inflammatory bowel disease, as used herein, can comprise any disease affecting the bowel and which involves a response from the immune system. In non-limiting example, inflammatory bowel disease can be Crohn's disease or ulcerative colitis.

Furthermore, increasing the concentration of the indoleamine 2,3-dioxygenase by increasing its expression, combined with a costimulatory blockade of T cell activation, can have a beneficial effect in reducing inflammation in inflammatory bowel disease in humans, thereby providing a new treatment approach. As used herein, the term “treatment” is intended to include at least a partial relief of symptoms of the disease up to and including complete abrogation of the disease. Treatment also includes preventing the development of the disease by administration of inducer compounds prior to the appearance of clinical symptoms or very early in the course of the disease before significant clinical symptoms and/or pathological changes have occurred.

A number of substances are known to increase the activity of indoleamine 2,3-dioxygenase in cells. Substances having this activity can be effective in treating inflammatory bowel disease and are within the scope of the present teachings. One class of such a substance comprises cytolytic T lymphocyte-associated antigen 4 (CTLA-4). A water soluble fusion protein comprising the sequence of CTLA-4, i.e., cytolytic T lymphocyte associated antigen 4-immunoglobulin (CTLA-4-Ig) has been shown to induce indoleamine 2,3dioxygenase in dendritic cells (Mellor et al., Journal of Immunology 171, 1652-1655, 2003; Grohmann et al., Nature Immunology 3, 1097-1101, 2002). Thus, both cytolytic T lymphocyte-associated antigen 4 immunoglobulin fusion polypeptide, cytolytic T lymphocyte-associated antigen 4, (with or without pegylation, discussed infra) or a combination thereof, can increase expression of the enzyme. These molecules can also mediate a costimulatory blockade. Accordingly, in various embodiments of the present teachings, administration of one or more of these molecules can diminish inflammation in inflammatory bowel disease (for commercial availability, see BD Biosciences, Pharmingen, San Diego, Calif.).

Interferon-gamma (IFN-γ) has also been shown to increase indoleamine 2,3 dioxygenase levels by increasing expression of the enzyme (Kudo et al., Molecular Human Reproduction 6, 369-374, 2000; Taylor et al., FASEB Journal 5, 2516-2522, 1991). Recombinant Human Interferon-gamma in lyophilized form is very water soluble and it is readily available commercially (see for example, BD Biosciences, Pharmingen, San Diego, Calif.).

Bacterial lipopolysaccharide has been shown to be an inducer of indoleamine 2,3 dioxygenase (see Fujigaki et al., European Journal of Immunology 31, 2313-1318, 2001; Lestage et al., Brain, Behavior, and Immunity 16, 596-601, 2002; Hwu et al. Journal of Immunology 164, 3596-3599, 2000). The inventors herein have also have shown bacterial lipopolysaccharide to be a weak inducer of indoleamine 2,3-dioxygenase. The lipopolysaccharide can be obtained from Enterobacteriaceae such as E. Coli or Salmonella species (for commercial availability, see Sigma-Aldrich, St. Louis, Mo.).

These and other substances can be used in the present teachings to increase indoleamine 2,3 dioxygenase levels, to promote a costimulatory blockade of T cell activation by antigen presenting cells of the gastrointestinal tract, or a combination of both increasing IDO levels and promoting a costimulatory blockade. In certain aspects of the present teachings, the substances can be pegylated, i.e. stably linked to polyethylene glycol, to obtain enhanced properties of solubility, stability, half-life and other pharmaceutically advantageous properties. Agents used in the present teachings for inducing IDO expression and/or blockading T cell costimulation include, in non-limiting example, pegylated CTLA-4-Ig and pegylated interferon-y. Methods and reagents for pegylation are disclosed in references such as U.S. Pat. No. 4,179,337 to Davis, and Francis et al., International Journal of Hematology 68,1-18, 1998.

In various embodiments of the present teachings, beneficial effects of administration of substances which induce indoleamine 2,3-dioxygenase and/or provide a costimulatory blockade of T cell activation in a patient with inflammatory bowel disease can include diminishment of inflammation that is not necessarily a full remediation of the disease. Thus, it is envisaged by the inventors herein that substances which induce indoleamine 2,3-dioxygenase and/or provide a costimulatory blockade of T cell activation can be used in treatment regimens that include one or more other pharmaceutical agents such as, in non-limiting example, 5-aminosalicylates (e.g., sulfasalazine, olsalazine, mesalazine or balsalazide), corticosteroids, azathioprine and the anti-tumor necrosis factor α monoclonal antibody infliximab (available commercially as Remicade® (Centocor, Inc., Malvern, Pa.)).

In various aspects of the present teachings, substances which increase activity of indoleamine 2,3-dioxygenase and/or provide a costimulatory blockade of T cell activation can be in pharmaceutically acceptable formulations or preparations. Such formulations are suitable for therapeutic use in patients following administration by any suitable route including local, topical, or systemic administration such as parenteral and oral routes of administration, and can include, for example, intraperitoneal, intravenous, subcutaneous, intramuscular, intranasal transdermal, and oral routes of administration. Administration can be either rapid as by injection or over a period of time as by slow infusion or administration of controlled release formulation. Accordingly, methods well known to skilled artisans can be used to determine a dosage and administration method that provides an immune tolerance-promoting amount of a ligand of B7 displayed on antigen presenting cells of the subject's gastrointestinal tract, for therapeutic effect in the treatment of inflammatory bowel disease. Such an amount can further include an indoleamine 2,3dioxygenase-inducing amount of a ligand of B7 comprised by antigen presenting cells of the subject's gastrointestinal tract. In non-limiting example, a skilled artisan can determine, using methods well known to skilled artisans, a therapeutically effective amount and administration route of a cytotoxic T lymphocyte-associated antigen 4-immunoglobulin for the treatment of inflammatory bowel disease. Both animal and human clinical studies, as well as general principles of pharmaceutical sciences, can provide guidance for determining treatment regimens (e.g., Lenschow et al., Science 257, 789-792, 1992; Kremer et al., New England Journal of Medicine 349, 1907-1915, 2003; Saha et al., Journal of Immunology 157, 3869-3875, 1996; van Elsas et al., Journal of Experimental Medicine 194, 481-489, 2001; Kita et al., Annals of Thoracic Surgery 75, 1123-1127, 2003); Srinivas, N. R. et al., Pharmaceutical Research 14, 911-916, 1997.

Compositions of the present teaching can be employed in the form of pharmaceutical preparations. Such preparations can be made using materials and methods well known in the pharmaceutical art. One non-limiting example of a preparation utilizes a vehicle of physiological saline solution, Other non-limiting examples of compositions can comprise other pharmaceutically acceptable carriers such as, for example, a physiological concentration of other non-toxic salts, an aqueous glucose solution (e.g., a 5% glucose solution), and/or sterile water. A suitable buffer also may be present in the composition. In various aspects, such compositions can be lyophilized and stored in a sterile ampoule, and reconstituted by the addition of sterile water. The primary solvent can be aqueous or alternatively non-aqueous. The substances can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Such excipients are those substances usually and customarily employed to formulate dosages for parenteral administration in either unit dosage or multi-dose form or for continuous or periodic infusion.

Dose administration can be repeated depending upon the pharmacokinetic parameters of the dosage formulation and the route of administration used.

It is also contemplated that certain formulations containing the substances of the present teachings are to be administered orally. Such formulations can be encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

In various embodiments, a specific dose can be calculated by a skilled artisan using well known principles. For example, calculations can be based upon the approximate body weight or body surface area of the patient or the volume of body space to be occupied. Another factor in considering the appropriate dose can be the particular route of administration selected. Further refinement of the calculations necessary to determine the appropriate dosage for treatment is routinely made by those of ordinary skill in the art. Such calculations can be made without undue experimentation by one skilled in the art. Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In various embodiments, the present teachings include packaged pharmaceuticals. The packaged pharmaceutical can comprise an immune tolerance-promoting amount of a ligand of B7 comprised by antigen presenting cells of the patient's gastrointestinal tract. An immune tolerance-promoting amount of a ligand of B7 can also comprise, in some configurations, an anti-inflammatory amount of an inducer that increases activity of indoleamine 2,3-dioxygenase in antigen presenting cells of a patient having inflammatory bowel disease. The substance can be in a pharmaceutically acceptable formulation. The packaged pharmaceutical can also include instructions for using said substance for treating inflammatory bowel disease in a patient. Such instructions can be in the form of a package insert, a pamphlet or a computer-readable form such as floppy disc, compact disc and the like.

The present teachings can be further understood by reference to the examples which follow.

### EXAMPLE 1

This example illustrates the cellular distribution of indoleamine 2,3-dioxygenase protein in the normal colon using immunohistochemistry.

We determined the presence of indoleamine 2,3-dioxygenase protein in lamina propria mononuclear cells and vascular endothelial cells in the colon as follows. The colons of SJL/J mice were removed and fixed in 10% formalin overnight and then transferred to 70% ethanol. After embedding in paraffin, 4 μm serial sections were prepared. Endogenous peroxidase was quenched for 30 minutes in 1% hydrogen peroxide/PBS. The sections were then treated with a solution of Nuclear Decloaker (Biocare Medical, Walnut Creek, Calif.) in a pressure cooker at 15 PSI for 3 minute and then with Avidin/Biotin blocking (Vector Lab., Burlingame, Calif.) for 20 minutes each. The sections were treated with Protein Block (Dako, Carpenteria, Calif.) for 10 minutes, and then incubated with indoleamine 2,3-dioxygenase primary antibody 1:100 at 30° C. for 1 hr.

To make the antibody, mouse indoleamine 2,3-dioxygenase cDNA was first obtained from mouse colon total RNA by reverse transcription and PCR amplification. The cDNA was cloned in the bacterial expression vector, pET28 a (Novagen, Madison Wis.), and recombinant indoleamine 2,3-dioxygenase protein was expressed in Novablue (DE3) cells (Novagen). Purified protein was isolated using His-binding affinity columns and used to immunize rabbits (Cocalico Biologicals, Reamstown, Pa.). Mouse indoleamine 2,3-dioxygenase antibody was purified by Protein-A Sepharose column chromatography separation of rabbit serum, followed by affinity purification with recombinant mouse indoleamine 2,3-dioxygenase. The secondary antibody, goat anti-rabbit biotinylated IgG (NEN Life Science, Boston, Mass.), was applied for 30 minutes 1:1000 at 30° C. The sections were incubated with SA-HRP (P0397, Dako, Carpenteria, Calif.) 1:1000 for 30 minutes at 30° C. and rinsed. DAB (D9015, Sigma, St. Louis, Mo.) was applied until staining was evident microscopically. The tissue sections were counterstained with hematoxylin.

Immunohistochemistry for detecting quinolinic acid protein was performed by perfusing mice with transcardial carbodiimide to form amide bonds between the carboxyl groups on quinolinic acid and the primary amines on tissue proteins. Tissues were removed and fixed in Bouin's solution. Anti-quinolinic acid antibodies were obtained by raising polyclonal antiserum, as described for indoleamine 2,3-dioxygenase but against quinolinic acid (quinolinic acid) conjugated to bovine thyroglobulin (as previously described by Moffett JR, Cell Tissue Research 278, 461-469, 1994). Anti-quinolinic acid antibodies were utilized for immunohistochemistry using the Vectastain Elite Kit (Vector Lab., Burlingame, Calif.) per manufacturer's instructions in conjunction with Avidin/Biotin blocking.

Immunostaining of tissues revealed the presence of indoleamine 2,3-dioxygenase protein in the endothelium of arteries in the lamina propria and the mesentery. Endothelial cells in veins did not express indoleamine 2,3-dioxygenase. Indoleamine 2,3-dioxygenase protein in lamina propria mononuclear cells was not detected using Bouin's-fixed or formalin-fixed sections; however, immunostaining of unfixed frozen sections revealed indoleamine 2,3-dioxygenase expression in a population of lamina propria cells with a morphology consistent with fibroblasts or dendritic cells. Cells with the same morphology and localization stain strongly for quinolinic acid, a product of tryptophan metabolism through indoleamine 2,3-dioxygenase.

These studies established that indoleamine 2,3-dioxygenase is expressed in the colon in arterial endothelial cells and a population of lamina propria mononuclear cells with the morphology of fibroblasts and/or dendritic cells. Not only was indoleamine 2,3-dioxygenase expressed in the normal colon lamina propria, but the protein itself was active as demonstrated by the presence of quinolinic acid, a metabolite of tryptophan through the kynurenine pathway, seen in cells with the same morphology as those expressing indoleamine 2,3-dioxygenase.

### EXAMPLE 2

This example illustrates the increase in indoleamine 2,3-dioxygenase in cells of the colon after treatment with Trinitrobenzene Sulfonic Acid.

Six week old female SJL/J mice weighing approximately 20 grams, which were maintained at a controlled temperature and light/dark cycle in a pathogen free facility, were anesthetized with an intraperitoneal injection of a 10% Ketamine/Xylazine mixture. Colitis was induced by intrarectal administration of 0.5 mg of Trinitrobenzene Sulfonic Acid in 35% ethanol via a flexible 3.5 Fr catheter inserted 4 cm proximal to the anus. Inhibition of indoleamine 2,3-dioxygenase was achieved by surgical insertion of slow release pellets comprising 1-methyltryptophan (1-mT, a competitive inhibitor of IDO) under the dorsal skin at the time of Trinitrobenzene Sulfonic Acid administration, whereas control mice received placebo pellets. The pellets released 1-mT at a constant rate of 0.9 mg/hr for a period of 10 days. Mice were sacrificed 4 days after treatment for determination of indoleamine 2,3-dioxygenase protein and mRNA levels.

Western blotting (Bio-Rad, Hercules, Calif.) for determining indoleamine 2,3-dioxygenase protein amount was performed on whole colon lysates obtained from the distal colons of both control mice and mice treated with Trinitrobenzene Sulfonic Acid. For Lamina Propria Mononuclear cells, 1×10⁶ cells were concentrated and loaded per lane. The samples were denatured and separated on an 8% Sodium Dodecyl Sulphate-Polyacrylamide (SDS-PAGE) gel. After electrophoresis, the separated proteins were then transferred to an Immobilon-P Transfer Membrane (Millipore, Be-dford, Mass.). Indoleamine 2,3-dioxygenase protein was detected with mouse indoleamine 2,3-dioxygenase primary antibody described in Example 1 above using ECL (Amersham). To make this antibody, mouse indoleamine 2,3-dioxygenase CDNA was first obtained from mouse colon total RNA by reverse transcription and PCR amplification. The cDNA was cloned in the bacterial expression vector, pET28 a (Novagen. Madison Wis.), and recombinant indoleamine 2,3-dioxygenase protein was expressed in Novablue (DE3) cells (Novagen). Purified protein was isolated using His-binding affinity columns and used to immunize rabbits (Cocalico Biologicals, Reamstown, Pa.). Anti-mouse indoleamine 2,3-dioxygenase antibody was purified by Protein-A Sepharose column chromatography separation of rabbit serum, followed by affinity purification with recombinant mouse indoleamine 2,3-dioxygenase. The secondary antibodies were donkey anti-rabbit linked to horseradish peroxidase. After probing for indoleamine 2,3-dioxygenase protein, the membranes were stripped and reprobed for β-actin, which was used in addition to the protein assay to ensure equal protein loading.

Real-Time PCR for determining indoleamine 2,3-dioxygenase mRNA amount was performed using primers designed for the mouse indoleamine 2,3-dioxygenase gene, as well as various cytokines using Primer Express Software. Primers were synthesized by the Protein and Nucleic Acid Chemistry Lab at Washington University. Total RNA was isolated from homogenized distal SJL/J mouse colon using Trizol per manufacturer's directions (Invitrogen, Carlsbad, Calif.). Reverse transcription was performed using random primers, dNTPs, and Superscript II (Invitrogen). Mouse c-DNA was then used to perform real-time PCR using SYBR Green PCR Master Mix (Applied Biosystems Foster City, Calif.) as the detection system in the i-Cycler (Bio-Rad) or the ABI PRISM 7000 Sequence Detection System (Applied Biosystems). The PCR products were validated by melt analysis.

Immunofluorescence for detecting indoleamine 2,3-dioxygenase protein was performed on fresh-frozen colon sections of control and Trinitrobenzene Sulfonic Acid-treated mice, which were prepared by freezing in TISSUE-TEK O.C.T. Compound (Miles, Elkhart Ind.). Sections were cut at 6 microns and washed in 95% ethanol. They were then blocked in TNB solution for 30 minutes. The primary anti-indoleamine 2,3-dioxygenase used was as described in Example 1 above. The secondary antibody was an anti-rabbit IgG conjugated to Rhodamine Red (Jackson Immuno-research, West grove, Pa.). DAPI (DAKO Corporation Carpinteria, Calif.) was then used for nuclear counterstaining.

Immunohistochemistry for detecting quinolinic acid protein was performed as described above in Example 1.

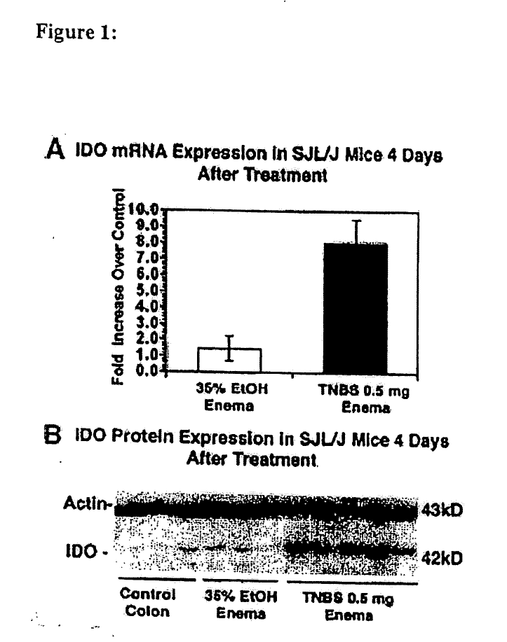

We determined whether the colitis induced in mice after treatment with Trinitrobenzene Sulfonic Acid is associated with increased amounts of indoleamine 2,3-dioxygenase mRNA and protein. Real-time PCR analysis of lysates from the distal colons of mice 4 days after treatment with Trinitrobenzene Sulfonic Acid showed a significant 8-fold increase in indoleamine 2,3-dioxygenase mRNA amounts when compared with untreated control mice (FIG. 1A). In mice receiving Trinitrobenzene Sulfonic Acid plus 1-mT, indoleamine 2,3-dioxygenase mRNA amounts were similar to that in mice receiving Trinitrobenzene Sulfonic Acid plus placebo (not shown). Administration of a 35% ethanol enema without Trinitrobenzene Sulfonic Acid had no significant effect on indoleamine 2,3-dioxygenase mRNA levels compared with control animals.

Induction of indoleamine 2,3-dioxygenase protein by Trinitrobenzene Sulfonic Acid administration was demonstrated by Western blotting techniques. A 42-kilodalton band representing indoleamine 2,3-dioxygenase was barely detectable in distal colon lysates from untreated mice. Treatment with a 35% ethanol enema alone did not induce indoleamine 2,3-dioxygenase protein amount. But, four days after Trinitrobenzene Sulfonic Acid administration, there was a marked induction of indoleamine 2,3-dioxygenase in the distal colon (FIG. 1 B). There was no difference in colonic indoleamine 2,3-dioxygenase protein amount between mice receiving Trinitrobenzene Sulfonic Acid plus placebo and those receiving Trinitrobenzene Sulfonic Acid plus 1-mT.

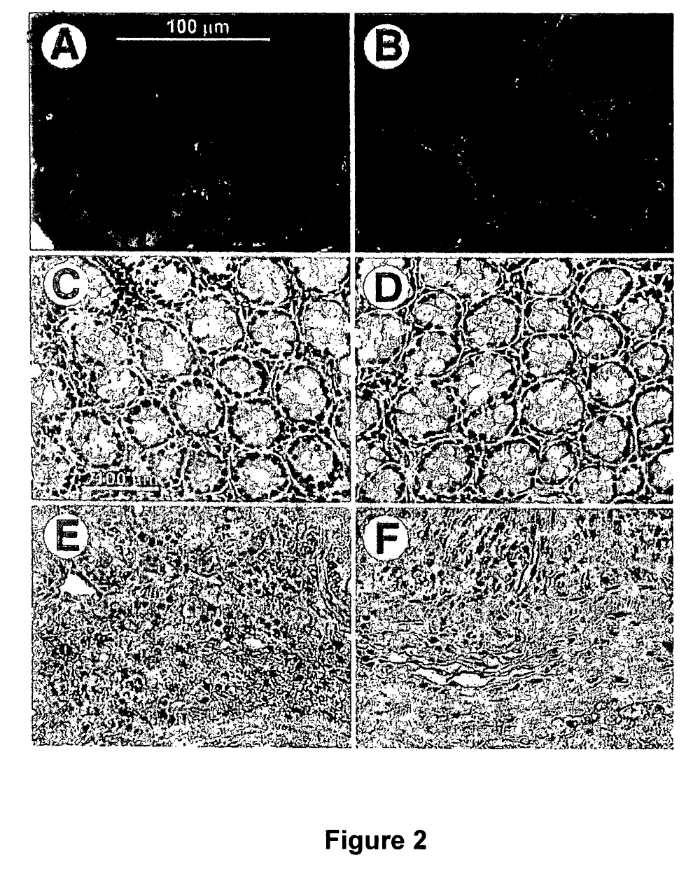

We also determined whether, in the setting of Trinitrobenzene Sulfonic Acid colitis, indoleamine 2,3-dioxygenase protein amount and activity are increased in colonic lamina propria mononuclear cells. Frozen sections of mouse colon demonstrated low baseline indoleamine 2,3-dioxygenase staining in the cytoplasm of mononuclear cells in the lamina propria surrounding the colonic crypts (FIG. 2A). In the setting of Trinitrobenzene Sulfonic Acid colitis, there was an increase in the staining intensity within individual cells as well as an increase in the number of staining cells (FIG. 2B). Lamina propria mononuclear cells with the same morphology and localization stained strongly for quinolinic acid, the product of tryptophan metabolism through indoleamine 2,3-dioxygenase (FIG. 2C and D). There was decreased quinolinic acid staining in the colons of mice treated with 1-mT versus placebo both in the presence and absence of Trinitrobenzene Sulfonic Acid colitis (FIG. 2C and F). This suggested diminished indoleamine 2,3-dioxygenase activity in the presence of this indoleamine 2,3-dioxygenase inhibitor.

These studies established that indoleamine 2,3-dioxygenase protein and mRNA amount are increased in the colon in the setting of Trinitrobenzene Sulfonic Acid colitis, a T helper 1 cell-mediated model. These studies further showed indoleamine 2,3-dioxygenase expression at baseline in lamina propria mononuclear cells in the colon, with a marked increase in amounts in these cells in Trinitrobenzene Sulfonic Acid colitis. Moreover, inhibition of indoleamine 2,3-dioxygenase with 1-mT resulted in decreased amounts of quinolinic acid in the colons of both untreated and Trinitrobenzene Sulfonic Acid-treated mice. Quinolinic acid is a catabolite of tryptophan through the kynurenine pathway; decreased quinolinic acid levels in the 1-mT-treated mice demonstrate that the drug achieved its predicted pharmacologic effect of inhibiting indoleamine 2,3-dioxygenase in the colon.

### EXAMPLE 3

This example illustrates the increase in mortality in Trinitrobenzene Sulfonic Acid colitis with indoleamine 2,3-dioxygenase inhibition.

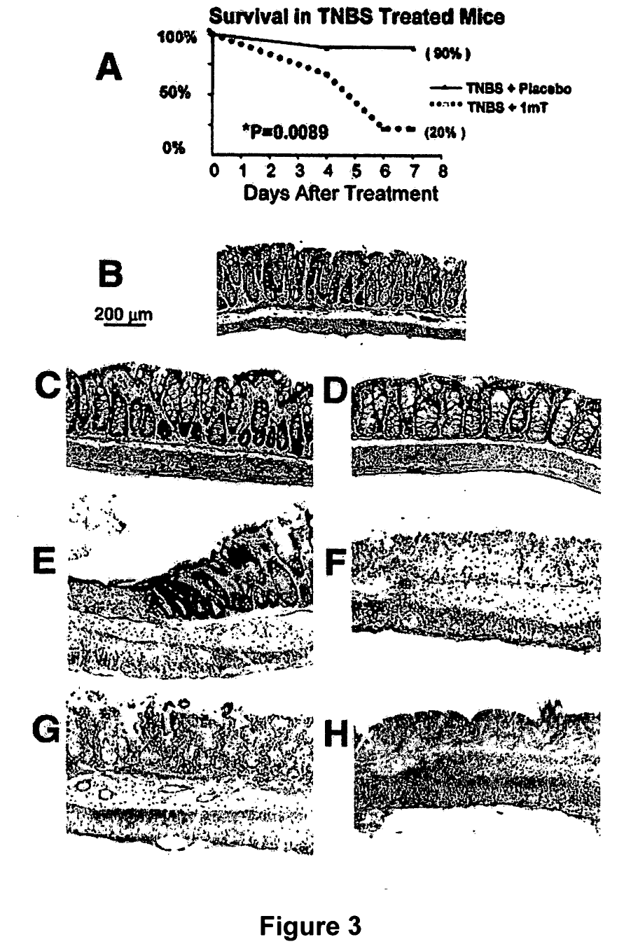

Mice received Trinitrobenzene Sulfonic Acid per rectum in addition to a subcutaneous pellet containing either placebo or 1-mT. Of the 10 mice that received Trinitrobenzene Sulfonic Acid plus 1-mT, 3 died within 4 days of Trinitrobenzene Sulfonic Acid administration. Of the remaining 7 mice, 5 developed tensely dilated stool-filled colons prior to death on days 4 to 6 after Trinitrobenzene Sulfonic Acid administration. Of the mice that received Trinitrobenzene Sulfonic Acid plus placebo, only 1 died, and none of the others developed significant dilation or stool retention. The survival rate was 100% in mice receiving a 35% ethanol enema along with either placebo or 1-mT (not shown). For mice receiving Trinitrobenzene Sulfonic Acid plus placebo, there was a 90% survival at 7 days after Trinitrobenzene Sulfonic Acid administration (FIG. 3A). In contrast, only 20% of the mice receiving Trinitrobenzene Sulfonic Acid plus 1-mT were still alive 7 days after Trinitrobenzene Sulfonic Acid treatment (FIG. 3A). Survival data were assessed using a χ² test. These studies demonstrated that inhibition of indoleamine 2,3-dioxygenase affects the course of the Th1-mediated Trinitrobenzene Sulfonic Acid model of colitis resulting in increased mortality.

### EXAMPLE 4

This example illustrates the effect of indoleamine 2,3-dioxygenase inhibition on gross morphology and histology in Trinitrobenzene Sulfonic Acid colitis.

The colon was removed from its mesentery to the pelvic brim by blunt dissection and the serosal surface examined under a dissecting microscope. The colon was then opened longitudinally along the mesenteric attachment and then pinned flat so that the mucosal surface could be examined. A modification of a scoring scale (Colon et al., Cytokine 15, 220-226, 2001) was used to assess the degree of macroscopic inflammation in the distal colon (1: normal mucosa; 2: edema and hyperemia; 3: small ulcers with mild intraperitoneal adhesions; 4: large ulcers [>7 mm]± extensive intraperitoneal adhesions; 5: megacolon, perforation, and necrosis).

The pinned out colon was then fixed in 10% formalin overnight and then transferred to 70% ethanol. After embedding in paraffin, 4 μm serial sections were prepared and stained with hematoxylin and eosin for histologic grading. A modification of the scoring scale of Fuss et al., Journal of. Immunology 168, 900-908, 2002 was used to assess the microscopic degree of inflammation on longitudinal sections of the colon (1: no evidence of inflammation; 2: low level of lymphocyte infiltration with infiltration seen in a <10% high-power field (hpf), no structural changes observed; 3: moderate lymphocyte infiltration with infiltration seen in 10% -25% hpf, crypt elongation, bowel wall thickening, which does not extend beyond mucosal layer, no evidence of ulceration; 4: high level of lymphocyte infiltration with infiltration seen in 25% -50% hpf, high vascular density, thickening of bowel wall, which extends beyond mucosal layer; 5: marked degree of lymphocyte infiltration with infiltration seen in >50% hpf, high vascular density, crypt elongation with distortion, transmural bowel wall-thickening with ulceration; 6: Complete loss of mucosal architecture (crypts) with ulceration covering >1 low-power field and loss of mucosal vasculature; 7: coagulation necrosis of at least the mucosal layer).

The histologic appearance of Trinitrobenzene Sulfonic Acid colitis was assessed in animals receiving 1-mT or placebo at 3 days, 4 days, and 6 days after Trinitrobenzene Sulfonic Acid administration. On day 3 after Trinitrobenzene Sulfonic Acid administration, there was no significant histologic differences between the 2groups (Table 1); however, after day 3, the histology in the Trinitrobenzene Sulfonic Acid plus placebo group began to improve, whereas that in the Trinitrobenzene Sulfonic Acid plus 1-mT group became progressively worse. The histologic scores in the group receiving Trinitrobenzene Sulfonic Acid plus 1-mT were significantly higher at day 4 and day 6. There was significant indoleamine 2,3-dioxygenase protein induction in colon lysates starting at around day 3 (not shown). Morphologic and histologic data were assessed using a Student t test.

Mice receiving 35% ethanol enemas demonstrated variable amounts of mucosal injury during the first 2 to 3 days after administration. At day 4, animals receiving a 35% ethanol enema and either placebo or 1-mT to inhibit indoleamine 2,3-dioxygenase demonstrated no ulceration or inflammatory infiltrate (FIG. 3C-D, respectively). There was no significant histologic difference between the 2 ethanol-treated groups and control animals (FIG. 3B) besides occasional goblet cell hyperplasia.

At day 4, animals receiving Trinitrobenzene Sulfonic Acid plus placebo developed areas of focal ulceration associated with transmural infiltration of inflammatory cells and thickening of the colonic wall, in particular in the muscularis (FIG. 3E). The affected area was limited to the region of the distal colon that had likely come into direct contact with Trinitrobenzene Sulfonic Acid. Except for some areas of focal ulceration, there was overall preservation of crypt architecture. By day 6, there was some improvement with fewer areas of focal ulceration (FIG. 3F).

At day 4 in the distal colons of mice receiving Trinitrobenzene Sulfonic Acid plus 1-mT, there was a loss of mucosal architecture and an increased transmural inflammatory infiltrate compared with the colons of mice receiving Trinitrobenzene Sulfonic Acid plus placebo (FIG. 3G). There was more uniform loss of epithelium with extensive circumferential ulceration. There was also a paucity of vessels within the lamina propria. By day 6, this region appears grossly necrotic with a lack of vascularity (FIG. 3H). There was progressive thinning of the colonic wall with persistence of an inflammatory infiltrate. The boundaries between the muscularis, submucosa, and mucosa became less obvious. In severely ill animals, there was complete loss of tissue architecture with evidence of perforation.

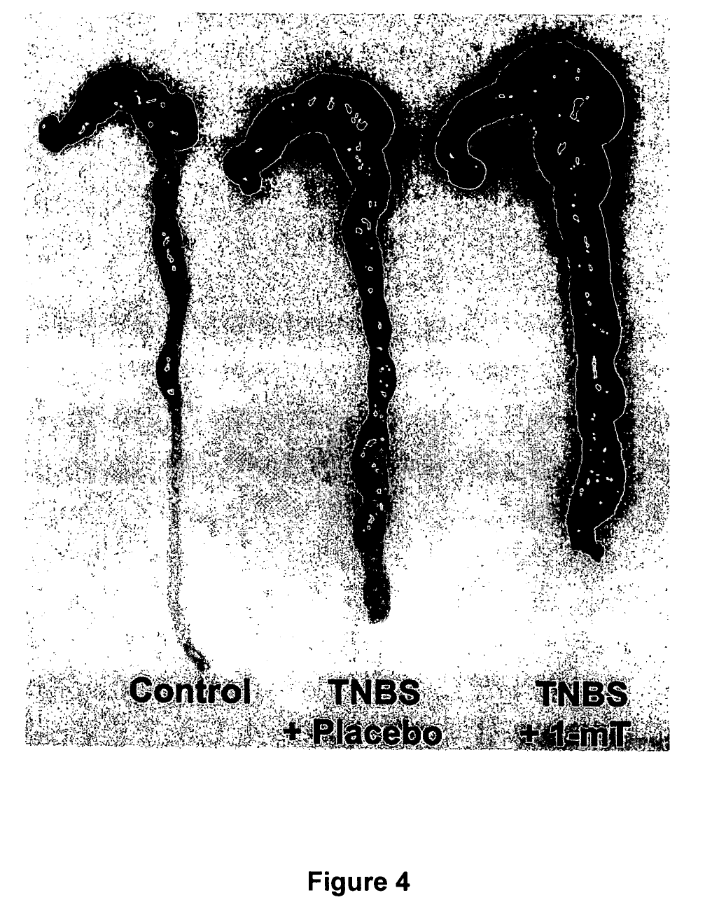

With respect to morphologic appearance of Trinitrobenzene Sulfonic Acid colitis, by day 4 after Trinitrobenzene Sulfonic Acid administration, there were significant gross morphologic differences between the colons of mice treated with Trinitrobenzene Sulfonic Acid plus 1-mT (to inhibit indoleamine 2,3-dioxygenase) and the colons of those treated with Trinitrobenzene Sulfonic Acid plus placebo (P=0.008, Table 1). At this time, there was gross evidence of inflammation and edema in the distal colons of both groups of animals (FIG. 4). However, the colons from the 1-mT-treated mice were significantly more indurated, dilated, and packed with solid stool. Similar to the histology described previously, there were no gross colonic morphologic differences between untreated control mice and mice receiving an ethanol enema with either 1-mT or placebo.

These studies demonstrated that administration of 1-mT had no effect on colonic morphology in untreated mice or in mice receiving 35% ethanol enemas in the absence of Trinitrobenzene Sulfonic Acid. Thus, pharmacologic inhibition of indoleamine 2,3-dioxygenase did not activate the mucosal immune system in the colon either in the absence of injury or in the presence of mild transient colonic injury as is seen with 35% ethanol enemas. However, inhibition of indoleamine 2,3-dioxygenase in mice with Trinitrobenzene Sulfonic Acid colitis resulted in a marked worsening of mucosal histology and increased mortality. The Trinitrobenzene Sulfonic Acid colitis model is a delayed type hypersensitivity response directed against TNP haptenated neoantigens, resulting in Th1 cell activation and, therefore, increased IFN-γ production. Histology in Trinitrobenzene Sulfonic Acid-treated mice receiving either placebo or 1-mT was similar during the first 3 days. Beginning at day 4, however, the histology in the Trinitrobenzene Sulfonic Acid- treated mice receiving placebo began to improve while that in the Trinitrobenzene Sulfonic Acid-treated mice receiving 1-mT continued to worsen. The timing of these events coincided with the appearance of increased indoleamine 2,3-dioxygenase expression typically seen by 3 days after Trinitrobenzene Sulfonic Acid administration. This was further evidence that indoleamine 2,3-dioxygenase was acting to antagonize the Th1 response to this potent immunologic stimulus.

### EXAMPLE 5

This example illustrates the increase in proinflammatory cytokine mRNA amounts within the colons of mice treated with Trinitrobenzene Sulfonic Acid and placebo or 1-mT using Real-Time PCR.

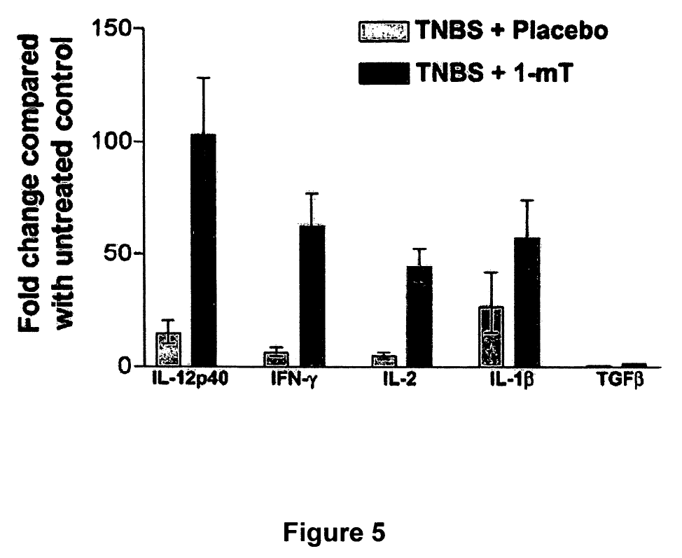

Primers were designed for the various cytokines using Primer Express Software and Real-Time PCR was performed as described in Example 1 above. Real-Time PCR was used to quantify mRNA expression of cytokines in colon lysates from mice treated with either placebo or 1-mT 4 days after Trinitrobenzene Sulfonic Acid administration. There was significant induction over baseline of the proinflammatory cytokines IL-12, IFN-γ, interleukin (IL)-2, and IL-1 in both Trinitrobenzene Sulfonic Acid-treated groups (FIG. 5). There was significantly higher expression by orders of magnitude of IL-12, IFN-γ, and IL-2 in mice receiving Trinitrobenzene Sulfonic Acid plus 1-mT-treated mice versus mice receiving Trinitrobenzene Sulfonic Acid and placebo-treated mice. IL-12 and IFN-γ expression increased 20-fold and 8-fold, respectively, in the placebo-treated group and 120-fold and 75-fold, respectively, in the 1-mT-treated group. In contrast, the expression of the anti-inflammatory cytokine TGFβ was about half of control values in the Trinitrobenzene Sulfonic Acid- and placebo-treated group and 1.5 times control values in the Trinitrobenzene Sulfonic Acid- and 1-mT treated group.

These studies established the increased levels of proinflammatory cytokines in the colons of mice receiving 1-mT in addition to Trinitrobenzene Sulfonic Acid, which may explain the worsening histology and increased mortality in these mice. IL-12, IFN-γ, IL-2, and IL-1 mRNA levels were all markedly increased in the animals receiving Trinitrobenzene Sulfonic Acid plus 1-mT as compared with those receiving Trinitrobenzene Sulfonic Acid alone. Thus, all of the Th1-associated proinflammatory cytokines that were increased in Trinitrobenzene Sulfonic Acid colitis were increased to a significantly greater degree in mice receiving 1-mT in addition to Trinitrobenzene Sulfonic Acid. Inhibition of indoleamine 2,3-dioxygenase enhanced Th1 immune activation by increasing levels of Th1-related cytokines; this implied that indoleamine 2,3-dioxygenase functions to down-regulate Th1-mediated inflammation. The increased levels of IFN-γ seen in mice receiving Trinitrobenzene Sulfonic Acid plus 1-mT suggested a feedback loop in which IFN-γ up-regulated indoleamine 2,3-dioxygenase expression and indoleamine 2,3-dioxygenase expression decreased IFN-γ production through inhibition of T-cell activation and proliferation.

### EXAMPLE 6

This example illustrates the increase in indoleamine 2,3-dioxygenase following treatment with recombinant IFN-γ.

In order to obtain the various colonic lamina propria cellular subpopulations, lamina propria mononuclear cells were isolated from mouse colon and then fractionated using magnetic immunoselection. To isolate lamina propria mononuclear cells, mice were sacrificed, and their colons removed and placed in ice-cold PBS. Intestines were opened along the mesenteric attachment and isolation was performed as described in Newberry, Journal of Immunology 166, 4465-4472, 2001. The isolated lamina propria mononuclear cells were then used for fractionation of lamina propria mononuclear cell subpopulations as described below or cultured in 96-well tissue culture plates at a density of 2.5×10⁶ cells/mL in RPMI 1640 medium (BioWhittaker, Walkersville, Md.) containing 2 mmol/L Glutamax I (L-Alanyl-L Glutamine; Life Technologies, Gaithersburg, MD), 10 mmol/L HEPES, 1 mmol/L sodium pyruvate, 50 U/mL penicillin-50 mg/mL streptomycin, 50 μmol/L βB-) mercaptoethanol, and 10% FCS (HyClone, Logan, Utah) at 37° C. and 5% CO2 in the presence or absence of LPS 30 ng/mL (Sigma) and/or recombinant interferon-γ (rIFN-γ) 30 ng/mL (R&D systems) for 24 hours at 37° C.

Fractionation of lamina propria mononuclear cells was performed using magnetic immunoselection. Colonic lamina propria mononuclear cells were resuspended at 2×10⁷ cells/mL in PBS with 1% bovine serum albumin (Fischer Scientific) and 1 mg/mL human IgG (Sandoz Pharmaceuticals, East Hanover, N.J.) for 20 minutes on ice. Cells were then incubated with biotin-conjugated anti-mouse B220 antibody (BD Biosciences) diluted in PBS containing 1% bovine serum albumin and 1 mg/mL human IgG for 20 minutes on ice, washed in PBS containing 1% BSA, incubated with streptavidin-conjugated microbeads (Miltenyi Biotech, Auburn, Calif.) per the manufacturer's directions, and magnetically sorted using MACS columns (Milteyni Biotech). B220-depleted cells were then incubated with a biotin-conjugated anti-MHC II antibody (BD Biosciences, catalog No. 553540), which is cross-reactive with the H-2^{s} haplotype, in PBS containing 1% bovine serum albumin and 1 mg/mL human IgG for 20 minutes on ice. B220-depleted cells were washed in PBS containing 1% BSA and then incubated with streptavidin-conjugated microbeads and magnetically sorted as noted previously. Isolated cells were cultured in 96-well plates at a density of 5×10⁶ cells/mL in the presence or absence of rIFN-γ and/or LPS as described above. An aliquot of each isolated cell population was cultured overnight at 37° C. and 5% CO2 in the above media to allow for the phagocytosis of the streptavidin-coated microbeads and then analyzed by flow cytometry with the following antibodies/reagents: fluoresecin isothiocyanate (FITC)-conjugated anti-CD45, phycoerythrin (PE)-conjugated anti-CD19, FITC-conjugated anti-MHCII, biotin-conjugated anti-CD11c, biotin-conjugated anti-TCR-β, streptavidin-conjugated PE, appropriate isotype control antibodies (all available from BD Biosciences), and biotin-conjugated anti-F480 (Cedarlane Laboratories, Hornby, Ontario, Canada). Studies have shown that inhibition of indoleamine 2,3-dioxygenase activity in professional antigen presenting cells (macrophages and dendritic cells) augments T-cell-mediated inflammatory responses. Therefore, inhibition of indoleamine 2,3-dioxygenase activity in professional antigen presenting cells in the colon may account for the worsened histology, mortality, and increased production of the inflammatory cytokines that we observed. Professional antigen presenting cells account for approximately 10% of the lamina propria mononuclear cell population. To effectively enrich for professional antigen presenting cells, we first depleted B220+ cells (primarily B-lymphocytes) and then selected for MHC II⁺ cells (primarily professional antigen presenting cells). This MHCII⁺/B220⁻ fraction is 4-fold enriched (40% of total cells) for professional antigen presenting cells compared with unfractionated lamina propria mononuclear cells. Half of these antigen presenting cells are CD11c positive dendritic cells and half are F4/80 positive macrophages.

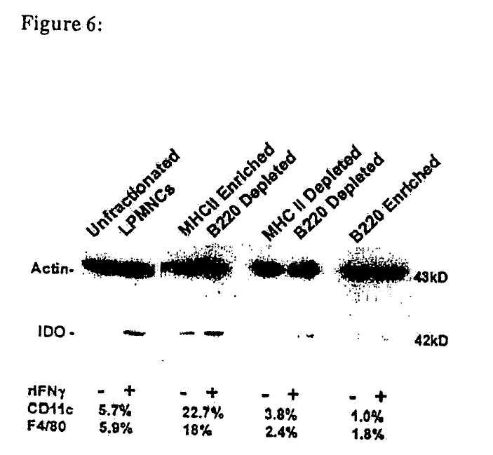

We determined whether indoleamine 2,3-dioxygenase is present in lamina propria mononuclear cell subpopulations isolated from colons of untreated mice and whether indoleamine 2,3-dioxygenase amounts increase when the cells are cultured with IFN-γ (FIG. 6). Indoleamine 2,3-dioxygenase was present in unstimulated lamina propria mononuclear cells; incubation with rIFN-γ resulted in a marked increase in indoleamine 2,3-dioxygenase expression. Indoleamine 2,3-dioxygenase was highly expressed at baseline in the MHCII⁺/B220⁻ lamina propria mononuclear cell population and was further induced in this population after incubation with rIFN-γ. Basal indoleamine 2,3-dioxygenase expression by this population was likely due to activation of these antigen presenting cells in situ because indoleamine 2,3-dioxygenase-expressing cells are seen in the colonic LP of unmanipulated animals (FIG. 2A). The B220⁺ enriched lamina propria mononuclear cell population did not express significant indoleamine 2,3-dioxygenase at baseline but did express indoleamine 2,3-dioxygenase after incubation with rIFN-γ. This population contained about 3% professional antigen presenting cells. The B220⁻-/MHCII⁻ lamina propria mononuclear cell population expressed an intermediate amount of indoleamine 2,3-dioxygenase at baseline, which increased after incubation with rIFN-γ. This population included 6% professional antigen presenting cells, 22% T cells, and 67% CD45⁻ cells, which include stromal cells and endothelial cells.

These studies establish that indoleamine 2,3-dioxygenase is highly present in lamina propria antigen presenting cells at baseline and is increased in amounts in multiple cell types after incubation with IFN-γ. In fractionated lamina propria mononuclear cells, indoleamine 2,3-dioxygenase expression at baseline was associated with B220⁻-/MHC II⁺ cells, a cell fraction that is enriched for professional antigen presenting cells including (dendritic cells and macrophages). Although B220⁻/MHC III⁺ cells make up only 10% of the lamina propria mononuclear cell population, they account for the majority of the total baseline indoleamine 2,3-dioxygenase expression. From these data, we conclude that incubation of this antigen presenting cell-rich population with rIFN-γ further induced indoleamine 2,3-dioxygenase expression.

### EXAMPLE 7

This example illustrates decreased indoleamine 2,3-dioxygenase expression and increased inflammation of the colon of STAT-1 and IFN-γ knockout mice in response to Trinitrobenzene Sulfonic Acid. STAT1⁻/⁻ mice are deficient for the STAT-1 transcription factor of the JAK-STAT signaling pathway, Meraz, M. A. et al., Cell 84, 431-442, 1996, while IFN-γ Receptor ⁻/⁻ mice are deficient for the interferon-gamma receptor, Kaplan, D. H., et al., Proceedings of the National Academy of Sciences USA 95, 7556-7561, 1998. It is believed that interferon-γ, upon binding to its receptor, activates STAT-1, see, e.g., Bromberg, J. F. et al., Proceedings of the National Academy of Sciences USA 93, 7673-7678, 1996.

STAT1⁻/⁻ and IFN-γReceptor ⁻/⁻ mice were on the C57BL/6 background. C57BL/6 mice approximately 6 to 8 weeks of age and matched for age and gender with the knockouts were purchased from the Jackson Laboratory.

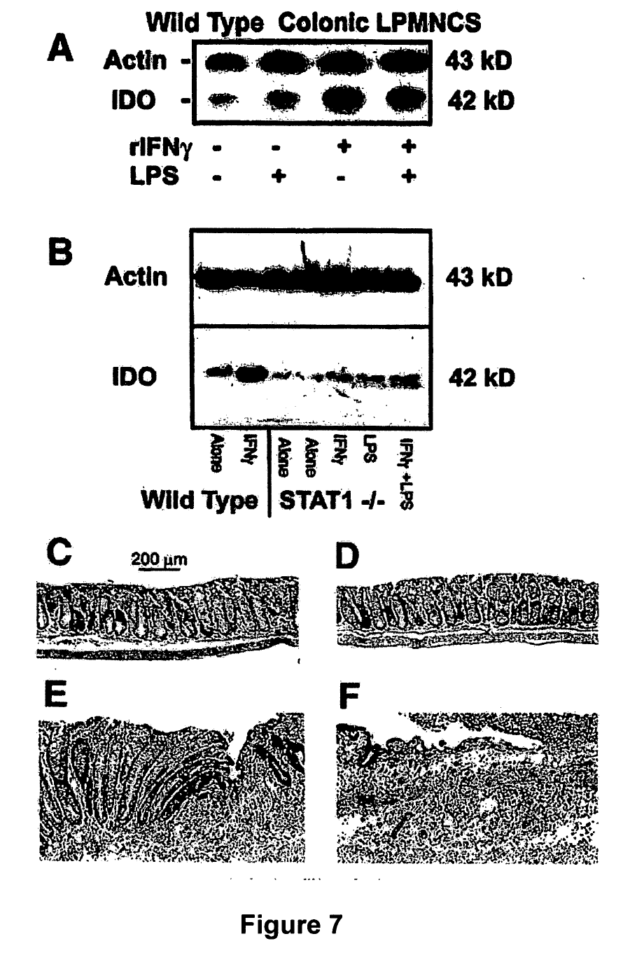

We determined whether STAT1⁻/⁻ mice had impaired lamina propria mononuclear cell indoleamine 2,3-dioxygenase induction in response to IFN-γ and develop a more severe form of Trinitrobenzene Sulfonic Acid colitis as compared with wild-type controls. There was baseline indoleamine 2,3-dioxygenase expression in isolated colonic lamina propria mononuclear cells from C57BL/6 mice. This expression increased significantly following a 24-hour incubation with rIFN-γ. Culturing these cells with LPS for 24 hours produced a relatively weak indoleamine 2,3-dioxygenase induction compared with IFN-γ alone. IFN-γ alone induced indoleamine 2,3-dioxygenase to the same extent as the combination of LPS and IFN-γ (FIG. 7A). But, in STAT1⁻/⁻ mice, they demonstrated impaired lamina propria mononuclear cell indoleamine 2,3-dioxygenase induction in response to r IFN-γ and developed a more severe form of Trinitrobenzene Sulfonic Acid colitis as compared with wild-type controls. Colon lamina propria mononuclear cells from STAT1⁻/⁻ animals had a severely blunted indoleamine 2,3-dioxygenase protein induction in response to IFN-γ (FIG. 7B) but not necessarily LPS. When STAT1⁻/⁻ mice were given 2 mg Trinitrobenzene Sulfonic Acid, they developed significantly more inflammation and distal colonic injury compared with control C57BL/6 mice given the same dose of Trinitrobenzene Sulfonic Acid (FIG. 7C-F).

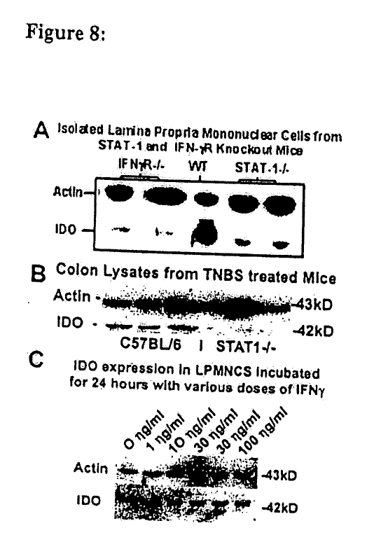

Furthermore, these studies showed that IFN-γ induction of indoleamine 2,3-dioxygenase was blunted in lamina propria mononuclear cells isolated from STAT1⁻/⁻ mice, which lack a signaling molecule necessary for IFN-γ responsiveness. IFN-γ Receptor ⁻/⁻ mice, like the STAT-1 ⁻/⁻ mice, had decreased basal indoleamine 2,3-dioxygenase protein (FIG. 8A). Distal colon lysates from control C57BL/6 mice with Trinitrobenzene Sulfonic Acid colitis demonstrated increased indoleamine 2,3-dioxygenase protein content, while indoleamine 2,3-dioxygenase protein was barely detectable in distal colonic lysates from STAT-1 ⁻/⁻ animals in the presence of Trinitrobenzene Sulfonic Acid colitis (FIG. 8B). Extremely low concentration (less than 1 ng/ml) of recombinant IFN-γ was required to maximally increase indoleamine 2,3-dioxygenase protein in lamina propria mononuclear cells (FIG. 8C). This was evidence that IFN-γ is essential for maximal indoleamine 2,3-dioxygenase expression because other known inducers (i.e. LPS) are not able to compensate for the IFN-γ unresponsiveness in the STAT1⁻/⁻ animals. Trinitrobenzene Sulfonic Acid colitis was more severe in STAT1⁻/⁻ mice than in wild-type controls, suggesting a protective role for IFN-γ in Trinitrobenzene Sulfonic Acid colitis via indoleamine 2,3-dioxygenase induction. Despite being considered a “proinflammatory” cytokine associated with Th1-mediated immune responses, there are several experimental colitis models in which IFN-γ appears to function in an anti-inflammatory manner. IFN-γ-deficient mice developed more severe crypt inflammation and colonic patch hypertrophy than do normal control animals in the setting of Trinitrobenzene Sulfonic Acid colitis. Also in the setting of Trinitrobenzene Sulfonic Acid colitis, colons from IFN-γ receptor deficient mice contained increased numbers of macrophages and CD4+ T cells, and their caudal lymph nodes produced increased levels of proinflammatory cytokines. These published studies, along with our data from STAT1⁻/⁻ animals, suggested an increased inflammatory response to Trinitrobenzene Sulfonic Acid in the absence of IFN-γ or its intracellular signaling pathway.

### EXAMPLE 8

This example illustrates that inhibiting indoleamine 2,3-dioxygenase in Trinitrobenzene Sulfonic Acid colitis increases lymphocyte proliferation.

Lymphocyte proliferation was determined by BrdU labeling of lamina propria lymphoid aggregates of mice treated with Trinitrobenzene Sulfonic Acid enemas plus subcutaneous pellets containing 1-methyl-tryptophan.

BrdU immunohistochemistry was performed to measure cellular proliferation in hypertrophied lymphoid aggregates within the lamina propria of Trinitrobenzene Sulfonic Acid ±1-methyl-tryptophan treated mice. Mice were given rectal Trinitrobenzene Sulfonic Acid and subcutaneous pellets containing 1-methyl-tryptophan or placebo. Groups of mice were sacrificed at 4 days and 6 days after Trinitrobenzene Sulfonic Acid administration. Each mouse received BrdU two hours prior to sacrifice in order to label the S-phase cells. Four micro paraffin sections were prepared from the inflamed colon segments with macroscopically visible hypertrophied colonic patches then labeling was performed as previously described in Riehl, Gasteroenterology, 118: 1106-16, 2000.

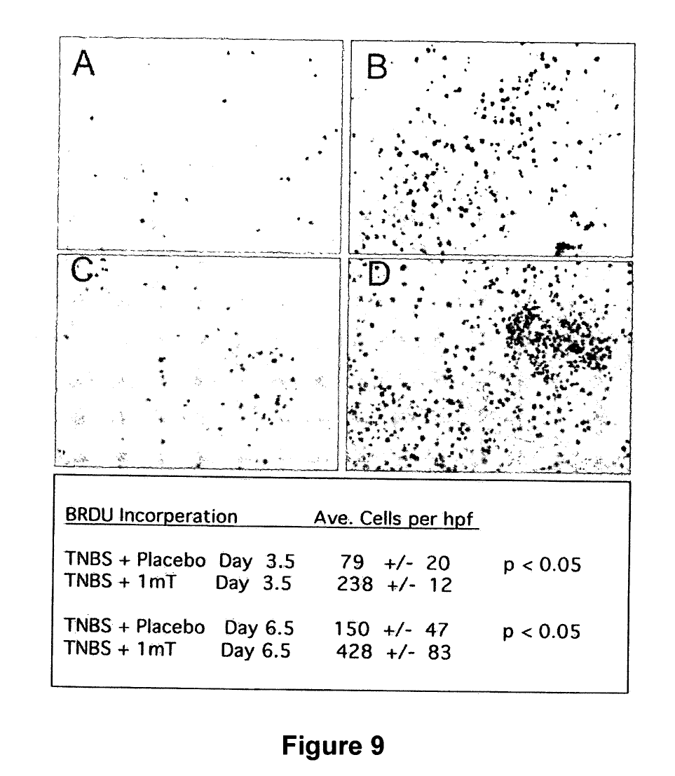

We determined that there were S phase lymphocytes in lymphoid aggregates from mice receiving Trinitrobenzene Sulfonic Acid plus placebo but significantly more S phase lymphocytes in lymphoid aggregates from mice receiving Trinitrobenzene Sulfonic Acid plus 1-methyl-tryptophan (FIG. 9). At each time-point shown, mice receiving Trinitrobenzene Sulfonic Acid plus 1-methyl-tryptophan demonstrated increased uptake of BrdU in mononuclear cells with a morphology consistent with lymphocytes. These studies showed that treatment with 1-methyl-tryptophan during Trinitrobenzene Sulfonic Acid colitis results in increased lymphocyte proliferation in colonic lymphoid aggregates. indoleamine 2,3-dioxygenase activity inhibits T cell proliferation and inhibition of indoleamine 2,3-dioxygenase with 1-methyl-tryptophan removes this block on T-cell proliferation. One consequence of this failure to down-regulate lymphocyte proliferation in the colon of 1-methyl-tryptophan treated animals was worsened colitis.

### EXAMPLE 9

This example illustrates the induction of indoleamine 2,3-dioxygenase by lipopolysaccharide (LPS) and cytotoxic T lymphocyte-associated antigen 4-immunoglobulin (CTLA-4-Ig) and the reduction of inflammation in colitis elicited by Trinitrobenzene Sulfonic Acid through systemic administration of LPS.

LPS (10 μg/mouse), an inducer of indoleamine 2,3-dioxygenase, was administered intraperitoneally to determine levels of indoleamine 2,3-dioxygenase and to determine the effect of LPS on cilitis.

To determine the effect of CTLA-4-Ig on indoleamine 2,3-dioxygenase, lamina propria mononuclear cells were cultured with various doses of CTLA-4-Ig, including 0, 10, 40, and 100 μg/ml.

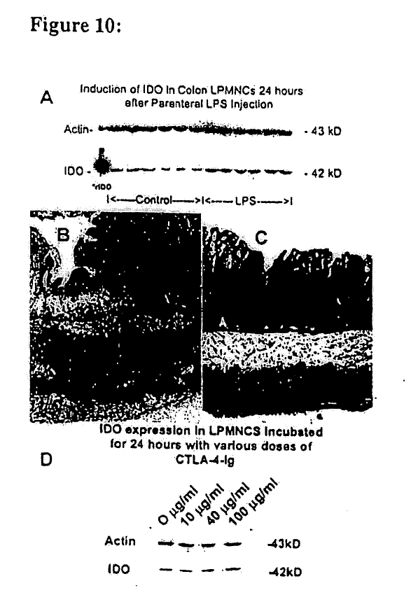

We determined whether systemic administration of LPS, an inducer of indoleamine 2,3-dioxygenase, in the days prior to Trinitrobenzene Sulfonic Acid administration, significantly reduced the inflammatory response to Trinitrobenzene Sulfonic Acid. Western blotting demonstrated a 3 to 4-fold increase in indoleamine 2,3-dioxygenase in lamina propria mononuclear cells isolated from SJL/J mice 24 hours after intraperitoneal administration of LPS (FIG. 10A). Furthermore, histological analysis demonstrated a markedly less inflammation in the LPS-treated mouse colon in response to Trinitrobenzene Sulfonic Acid (FIG. 10B and C). Also, we demonstrated that indoleamine 2,3-dioxygenase is increased in lamina propria cells cultured with various doses of CTLA-4-Ig, the maximal increase being achieved at a CTLA-4-Ig dose of 40 μg/ml (FIG. 10D).

These studies establish that increasing indoleamine 2,3-dioxygenase by administration of LPS prior to Trinitrobenzene Sulfonic Acid administration significantly diminished the resulting colitis. These studies further demonstrate that administration of CTLA-4-Ig can lead to an increase in indoleamine 2,3-dioxygenase in lamina propria cells.

### EXAMPLE 10

This example demononstrates that CTLA-4-Ig can clinically ameliorate Trinitrobenzene Sulfonic Acid (TNBS) Colitis.

In this and subsequent examples, Six week old female SJL/J mice weighing approximately 20 g were purchased from the Jackson Laboratory (Bar Harbor, Me.). All animals were maintained at a controlled temperature and light/dark cycle in a specific pathogen free facility at Washington University School of Medicine. The animals were treated in accordance with the NIH guidelines and our approved animal studies protocol. TNBS colitis was induced by intrarectal administration of 0.5 mg of TNBS (Sigma, St. Louis, Mo.) in 35% ethanol via a flexible 3.5 Fr catheter inserted 4 cm proximal to the anus (Neurath et al., Journal of Experimental Medicine 182, 1281-1290, 1995). CTLA-4-Ig (Sigma, St. Louis Mo.) 100 μg/mouse was administered via I.P. injection 24 and 6 hours prior to and 48 hours after TNBS administration. Pellets containing slow release 1-mT (Innovative Research of America, Sarasota, Florida) were surgically inserted under the dorsal skin of certain mice at the time of TNBS administration as described in Example 2, supra and in Gurtner et al., Gastroenterology 125, 1762-1773, 2003. Surviving mice were sacrificed at day 4 to assess morphological and histological differences and to obtain tissues for analysis.

For morphological and histological analysis, colons were removed from the mesentery to the pelvic brim by blunt dissection. Each colon was then opened longitudinally along the mesenteric attachment and then pinned flat so that the mucosal surface could be examined. The pinned out colon was then fixed in 10% formalin overnight and then transferred to 70% ethanol. After embedding in paraffin, 4-μm serial sections were prepared and stained with hematoxylin and eosin for histologic grading. The method for scoring histology was that described in Example 4, supra and in Gurtner, G. J. et al., Gastroenterology 125, 1762-1773, 2003. Morphological and histological data was assessed using a Student's t test. Survival data were assessed using a Chi square test. Fold increase in mRNA expression over control was assessed using a Student's t test.

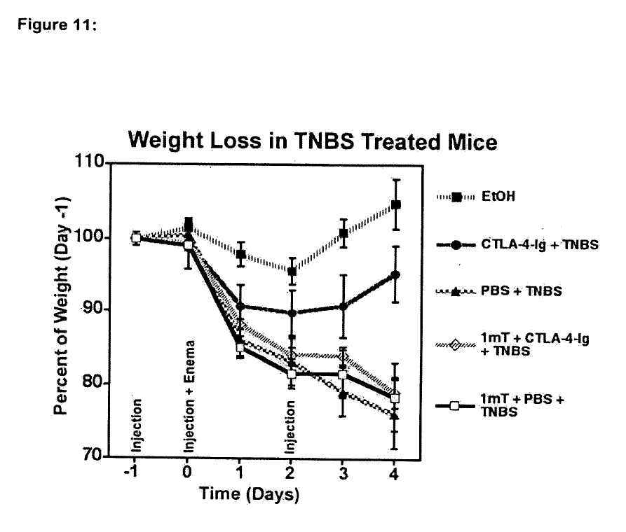

Weight loss, survival, and overall physical appearance are clinical markers for the severity of colitis in a TNBS model of Crohn's disease. All mice including the control animals that received a 35% EtOH enema (the vehicle for TNBS administration) had an initial weight loss associated with a diminished level of activity. As shown in FIG. 11, mice were treated with PBS or CTLA-4-Ig on days −1, 0, and 2 and received an enema of either EtOH or TNBS on day 0. Some mice also received a subcutaneous pellet of 1-mT on day 0. Surviving mice were sacrificed on Day 4. Mice receiving CTLA-4-Ig+TNBS (n=8) had an initial weight loss, and recovery that paralleled EtOH treated animals (n=4). CTLA-4-Ig+TNBS treated mice had significantly less weight loss than surviving animals treated with PBS+TNBS (n=9)(p<0.05) which had persistent weight loss through Day 4. However, mice receiving 1-mT in addition to CTLA-4-Ig+TNBS (n=8) also had persistent weight loss similar to surviving mice receiving PBS+TNBS and PBS+TNBS+1-mT (n=5). By the third day, however, the control animal's weight returned to baseline, as did their level of activity. Mice treated with the TNBS enema as well as systemic CTLA-4-Ig had a clinical course that paralleled that of the EtOH treated controls with a delayed recovery. These animals also began to gain weight at day three and by day four had a normal level of activity. In contrast, Mice receiving TNBS+PBS instead of CTLA-4-Ig continued to lose weight throughout the duration of the experiment. By Days 3 and 4 there was a statistically significant difference in weight change between these two TNBS treatment groups (p<0.05).

As shown in FIG. 11, the gain in weight seen in animals receiving CTLA-4-Ig+TNBS could be reversed through IDO inhibition with 1-mT. Instead of recovering weight, CTLA-4-Ig+TNBS-treated animals also receiving 1-mT had a persistent trend in weight loss comparable to animals treated with PBS+TNBS. By Day 4 there was also a statistical difference in weight change between CTLA-4-Ig+TNBS treated animals and those also receiving 1-mT (p<0.05). These data indicate that administration of CTLA-4-Ig can clinically alleviate symptoms in an animal model of inflammatory bowel disease.

### EXAMPLE 11

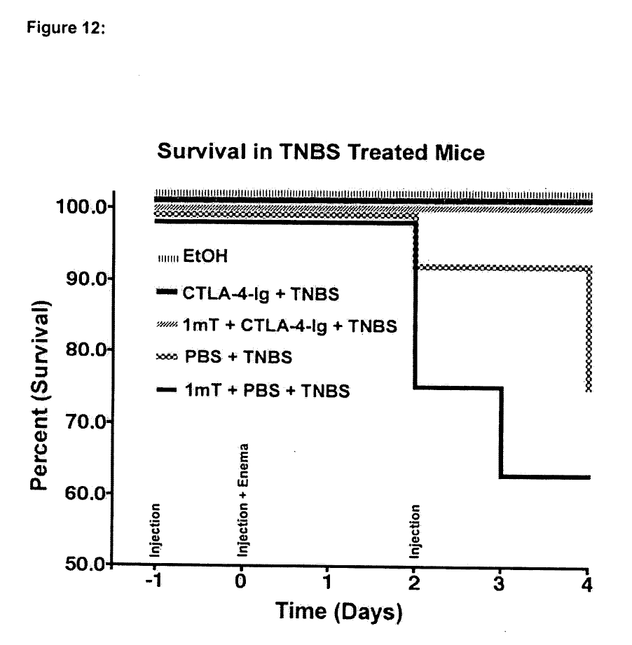

This example illustrates survival in CTLA-4-Ig and TNBS-treated mice. These experiments utilized materials and methods as described in Example 10. As shown in FIG. 12, mice receiving CTLA-4-Ig with TNBS had a 100% survival rate (N=16) regardless of IDO inhibition. Animals treated with TNBS+CTLA-4-Ig+1-mT had no mortality (0 of 8) by Day 4, which is identical to CTLA-4-Ig+TNBS treated animals (0 of 8) as well as EtOH enema treated controls (0 of 4). This survival rate is identical to EtOH treated animals and is significantly better than either the PBS+TNBS (9/12 or 75%) or PBS+TNBS+1-mT (5/8 or 62.5%) treated groups. Animals that received CTLA-4-Ig along with TNBS had statistically less mortality than TNBS treated animals not receiving CTLA-4-Ig. PBS+TNBS treated animals had a 25% mortality rate (3 of 12) and this increased to 37% (3 of 8) with the addition of 1-mT to inhibit IDO (p<0.05 for both). These data indicate that CTLA-4-Ig can promote survival of mice presenting an animal model of inflammatory bowel disease.

### EXAMPLE 12

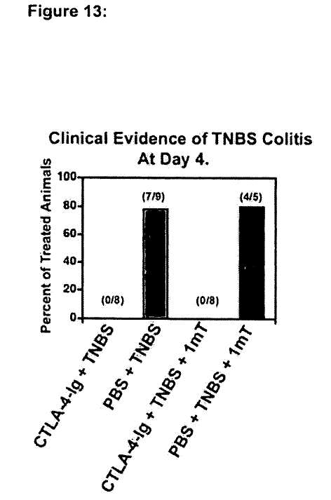

This example illustrates clinical characteristics of CTLA-4-Ig and TNBS treated mice. These experiments utilized materials and methods as described in Example 10. As shown in FIG. 13, CTLA-4-Ig administration can abrogate many of the clinical characteristics of TNBS colitis. In the experiments presented, mice receiving CTLA-4-Ig had an almost complete abrogation of many of the clinical characteristics of TNBS colitis regardless of 1-mT administration. 78% (7/9) of surviving mice receiving TNBS+PBS, but not CTLA-4-Ig, developed characteristics of active TNBS colitis. In contrast, none (0/8) of the CTLA-4-Ig+TNBS treated animals had these particular characteristics. Likewise, none (0/8) of the CTLA-4-Ig+TNBS treated animals appeared clinically ill even with the addition of 1-mT to inhibit IDO. However, 80% (4/5) of surviving animals treated with TNBS+PBS+1-mT had clinical characteristics of TNBS colitis. 78% (7/9) of surviving mice receiving TNBS+PBS, but not CTLA-4-Ig, developed a hunched over appearance with piloerection that is characteristic of active TNBS colitis. None (0/8) of the CTLA-4-Ig+TNBS treated animals had these particular characteristics. Likewise, none (0/8) of the CTLA-4-Ig+TNBS treated animals appeared clinically ill even with the addition of 1-mT to inhibit IDO. However, 80% (4/5) of surviving animals treated with TNBS+PBS+1-mT appeared hunched and ruffled with a marked decline in physical activity. Accordingly, CTLA-4-Ig administration can ameliorate clinical symptoms of inflammatory bowel disease.

### EXAMPLE 13

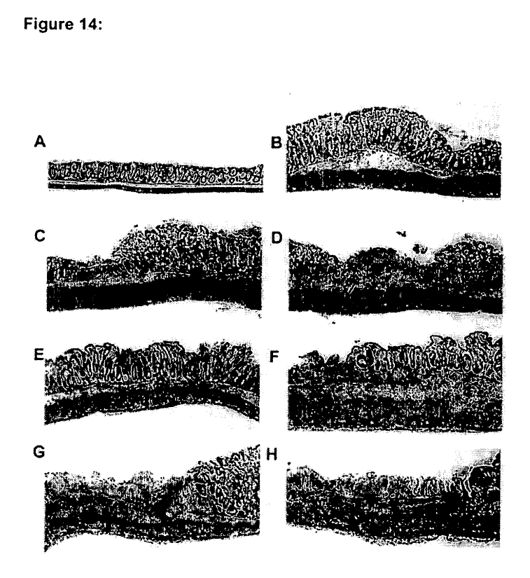

This example illustrates that CTLA-4-Ig can ameliorate TNBS colitis by morphological and histological criteria. These experiments utilized materials and methods as described in Example 10. As shown in FIG. 14, systemic CTLA-4-Ig administration was protective in the setting of TNBS colitis.

FIG. 14A illustrates normal appearing distal colon of a mouse treated with CTLA-4-Ig and an ethanol enema at Day 2. Systemic administration of CTLA-4-Ig had no obvious effect on colon histology in untreated animals or in control animals treated with EtOH enemas.

FIG. 14B shows crypt elongation, submucosal edema, and muscular wall thickening in the distal colon of a mouse treated with CTLA-4-Ig+TNBS at Day 4. For the most part, animals treated with CTLA-4-Ig+TNBS generally had mild disease characterized by crypt elongation, edema and muscular wall thickening with very focal areas of mild ulceration. These data show that systemic CTLA-4-Ig administration can minimize areas of focal ulceration and mucosal damage. However, this could be reversed through IDO inhibition.

FIG. 14C illustrates mild mucosal inflammation with mild ulceration and submucosal edema in the distal colon of a mouse treated with CTLA-4-Ig+TNBS at Day 2. These data demonstrate that two days after TNBS administration there was minimal ulceration and minimal inflammatory infiltrate in the CTLA-4-Ig treated animals.

FIG. 14D illustrates more extensive ulceration and inflammatory infiltration into the lamina propria and submucosa, as well as increased mucosal necrosis in a PBS+TNBS treated mouse also at Day 2. The PBS treated animals had more extensive mucosal damage and ulceration with increased amounts of lamina propria and submucosal inflammatory infiltration.

FIG. 14E illustrates that the mucosal damage and ulceration observed in FIG. 14D became more apparent at Day 4. Minimal lamina propria and submucosal inflammatory infiltrate, edema, and ulceration in a mouse treated with CTLA-4-Ig+TNBS were observed at Day 4. The CTLA-4-Ig treated animals had very localized disease with minimal inflammatory infiltrate, edema and ulceration compared to the PBS treated animals.

FIG. 14F illustrates that on Day 4, PBS+TNBS treated animals had significantly larger areas of ulceration and edema, and had significantly more transmural inflammation extending beyond the serosal surface.

FIG. 14G illustrates that histological abrogation of colitis seen in the CTLA-4-Ig treated animals can be reversed by IDO inhibition with 1-mT. Animals treated with CTLA-4-Ig+TNBS+1-mT had histological colitis that was comparable or more severe than TNBS+PBS treated animals. These animals had transmural inflammation with a loss of mucosal architecture and vasculature that was similar but less severe than animals receiving PBS+TNBS+1-mT. Extensive ulceration, increased trans-mural inflammation with a significant loss of mucosal architecture and vasculature are seen in a CTLA-4-Ig+TNBS+1-mT treated mouse at Day 4.

FIG. 14H illustrates that surviving animals treated with TNBS+PBS+1-mT generally had more transmural inflammation, loss of tissue architecture and vasculature, and outright necrosis. Extensive ulceration, necrosis, and extreme trans-mural inflammation are observed in the distal colon of a mouse treated with PBS+TNBS+1-mT at Day 4.

### EXAMPLE 14

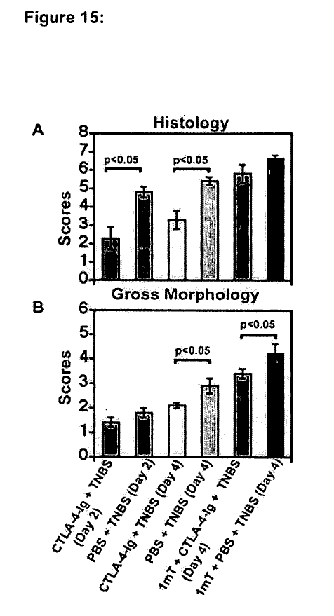

This example illustrates amelioration by CTLA-4-Ig of TNBS colitis by histological scoring, and this abrogation could be reversed through indoleamine 2,3-dioxygenase inhibition with 1-mT. These experiments utilized materials and methods as described in Example 10. FIG. 15A illustrates that CTLA-4-Ig ameliorated TNBS colitis as assessed through histological scoring, and this abrogation could be reversed through IDO inhibition with 1-mT. Scoring in Animals treated with TNBS+CTLA-4-Ig+1-mT was not significantly different than either TNBS+PBS or TNBS+PBS+1-mT treated animals at Day 4. At both Days 2 and 4 of TNBS treatment, CTLA-4-Ig treated animals had significantly lower histological scores than PBS treated animals [2.3 (n=4) vs 4.8 (n=4) and 3.3 (n=7) vs 5.4 (n=8) respectively p<0.05 for both]. At Day 4 of TNBS, PBS treated animals scored significantly lower than animals also treated with 1-mT to inhibit IDO [5.4 (n=8) vs. 6.6 (n=5) respectively p<0.05]. However animals treated with TNBS+CTLA-4-Ig+1-mT did not score significantly differently from either PBS treated or PBS+1-mT treated animals also receiving TNBS [5.8 (n=6)].

FIG. 15B illustrates that CTLA-4-Ig ameliorated TNBS colitis by morphological scoring only at Day 4. The data indicate that addition of 1-mT reversed the protective effect of CTLA-4-Ig, however, scoring was still significantly lower in TNBS+CTLA-4-Ig+1-mT treated animals than in TNBS+PBS+1-mT treated animals. At Day 2, there was no statistical difference in gross morphology between animals receiving TNBS and either CTLA-4-Ig or PBS. However, there was markedly less gross eschar formation on the mucosal surface of the CTLA-4-Ig treated animals (Not shown), but this was not figured into the scoring system. At Day 4 of TNBS treatment, CTLA-4-Ig treated animals had significantly lower morphological scores than PBS treated animals [2.1 (n=8) vs. 2.9 (n=9) respectively p<0.05]. This included significantly less edema, induration, mucosal ulceration, intra-peritoneal adhesions, and discoloration.

FIG. 15B further indicates that 1-mT could reverse the abrogation in CTLA-4-Ig treated animals at Day 4. CTLA-4-Ig+TNBS treated animals receiving 1-mT also had significantly higher morphological scores than CTLA-4-Ig+TNBS treated animals not receiving 1-mT [3.4 (n=8) vs. 2.1 (n=8) respectively p<0.05] TNBS+PBS+1-mT treated animals also scored significantly higher than animals treated with TNBS+PBS [4.2 (n=5) vs 2.9 (n=9) respectively p<0.05]. Surviving animals treated with 1-mT+TNBS+PBS tended to be dilated, gangrenous, and showed a significant propensity to develop adhesions. Animals treated with 1-mT+TNBS+CTLA-4-Ig had comparable colonic induration, adhesions, and discoloration but significantly less colonic dilation than animals receiving 1-mT+PBS+TNBS accounting for this difference [3.4 (n=8) vs. 4.2 (n=5) respectively p<0.05]. The data of Examples 13 and 14 indicate that CTLA-4-Ig can abrogate symptoms of an inflammatory bowel disease such as TNBS colitis.

### EXAMPLE 15

This example illustrates that CTLA-4-Ig induces IDO in cultured lamina propria mononuclear cells.

We have established that lamina propria antigen presenting cells expressed the highest levels of IDO protein at baseline. We also showed that this expression increased after incubation with r IFN-γ (Examples 1 and 2, supra; Gurtner et al. Gastroenterology 125, 1762-1773, 2003). Therefore we sought to demonstrate increased IDO expression in isolated lamina propria mononuclear cells (LPMNCs) in response to culturing with various doses of CTLA-4-Ig for 24 hours. In these experiments, LPMNCs were isolated from mouse colon and cultured with three escalating doses of CTLA-4-Ig. Western blotting for IDO was then performed, as described below. In these experiments, mice were sacrificed and their colons removed and placed in ice-cold PBS. Colons were opened along the mesenteric attachment and isolation was performed as previously described (Newberry et al., Journal of Immunology 166, 4465-4472, 2001). The isolated LPMNCs were then used for fractionation of LPMNC subpopulations as described in Example 6, supra, or cultured in 96-well tissue culture plates at a density of 2.5×10⁶ cells/ml in RPMI 1640 medium (BioWhittaker, Walkersville, Md.) containing 2 mM Glutamax I (L-Alanyl-L Glutamine; Life Technologies, Gaithersburg, Md.), 10 mM HEPES, 1 mM sodium pyruvate, 50 U/ml penicillin-50 mg/ml streptomycin, 5 μM β-mercaptoethanol, and 10% FCS (HyClone, Logan, Utah) at 37° C. and 5% CO2 in the presence or absence of CTLA-4-Ig at 10, 40 and 100 μg/ml for 24 hours at 37° C.

Western blotting for indoleamine 2,3-dioxygenase was performed as follows. Protein assays (BIO-RAD, Hercules, Calif.) were performed on whole colon lysates obtained from the distal colons of both control mice and mice treated with TNBS. For LPMNCs, 1×10⁶ cells were concentrated and loaded per lane. The samples were denatured and separated on an 8% SDS-PAGE gel. Following electrophoresis, the separated proteins were transferred to an Immobilon-P Transfer Membrane (Millipore, Bedford, Mass.). The primary antibody used was a rabbit anti- murine IDO antibody as described in Example 2, supra, and the secondary antibody was donkey anti-rabbit linked to horseradish peroxidase (Amersham Pharmacia Biotech, UK). The protein was detected using ECL (Amersham). The membranes were then stripped and re-probed for β-Actin, which was used in addition to the protein assay to ensure equal protein loading (Santa Cruz Biotechnology).

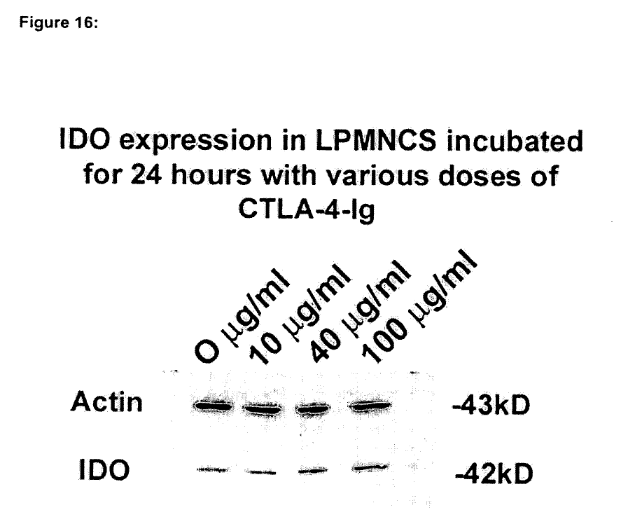

As illustrated in FIG. 16, Basal IDO expression was seen in control LPMNCs cultured in the absence of CTLA-4-Ig. Culturing LPMNCs with CTLA-4-Ig at a dose of 10 μg/ml had no detectable effect on IDO protein expression. However, there was a distinct increase in IDO protein expression in LPMNCs cultured with 40 μg/ml of CTLA-4-Ig and above. IDO expression did not increase further with higher doses of CTLA-4-Ig (i.e., 100 μg/ml). These data indicate that CTLA-4-Ig administration can promote an increase in expression of indoleamine 2,3-dioxygenase in cultured lamina propria mononuclear cells.

### EXAMPLE 16

This example illustrates that CTLA-4-Ig induces Colonic IDO and IFN-γ expression. Using quantitative real time PCR, we wished to assess colonic IDO mRNA expression in response to various experimental conditions involving CTLA-4-Ig administration in the setting of TNBS colitis. For the experiments, real time PCR was conducted as follows. Primers were designed for multiple genes using Primer Express Software (Applied Biosystems Foster City, Calif.). Primers were synthesized by the Protein and Nucleic Acid Chemistry Lab at Washington University. Total RNA was isolated from homogenized distal SJL/J mouse colon using Trizol per manufacturer's directions (Invitrogen, Carlsbad, Calif.). Reverse transcription was performed using random primers, dNTP's, and Superscript II (Invitrogen). Mouse c-DNA was then used to perform real Time PCR using SYBR Green PCR Master Mix (Applied Biosystems Foster City, Calif.) as the detection system in the ABI PRISM 7000 Sequence Detection System (Applied Biosystems). The PCR products were validated by melt analysis.

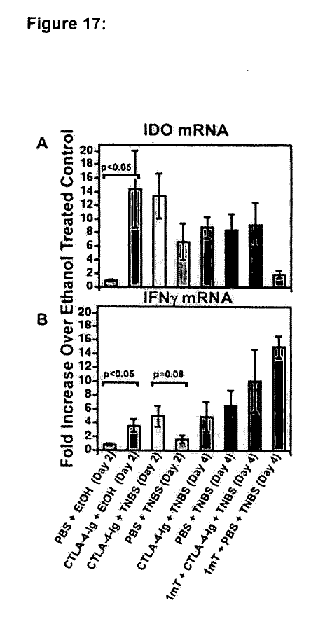

The results of PCR analyses are illustrated in FIG. 17. As shown in FIG. 17A, in control animals receiving EtOH enemas instead of TNBS, systemic CTLA-4-Ig administration induced a significant 15 fold increase in colonic IDO mRNA expression relative to PBS treated animals. CTLA-4-Ig administration also induced a 13 fold increase in IDO mRNA expression in TNBS treated animals at Day 2. This was twice that of PBS+TNBS treated animals which had a 7 fold increase in IDO expression at this same time point. By Day 4 of TNBS colitis, however, IDO expression in CTLA-4-Ig treated animals was 9 fold increased and comparable to that occurring in PBS treated animals that had an 8 fold increase in expression. In the setting of TNBS colitis and 1-mT administration, IDO expression was sustained in CTLA-4-Ig treated animals at a 9 fold increase but dropped off in PBS treated animals to a 2 fold increase over controls.

CTLA-4-Ig mediated induction of IDO expression in splenic dendritic cells requires autocrine or paracrine IFN-γ signaling (Finger et al., Nature Immunology 3, 1056-1057, 2002; Fallarino et al., Nature Immunology 4, 1206-1212, 2003; Grohmann et al., Nature Immunology 3, 1097-1101, 2002). Therefore we assessed IFN-γ expression by quantitative real time PCR, to determine if IFN-γ induction was required for IDO induction in the colon. As shown in FIG. 15B, CTLA-4-Ig administration was associated with significant 4 to 5 fold induction of IFN-γ in both control and TNBS treated animals However, at Day 2, there was a less than 2 fold increase in IFN-γ in PBS+TNBS treated animals. By Day 4, however, there was a 7 fold induction of IFN-γ mRNA while animals receiving TNBS continued to have a 5 fold induction. Also at Day 4 of TNBS colitis, 1-mT administration was associated with a 10 fold induction of IFN-γ expression in CTLA-4-Ig treated mice while PBS treated mice had a 15 fold increase. These data indicate that CTLA-4-Ig administration can lead to an increase in expression of both colonic IDO and IFN-γ.

### EXAMPLE 17

This example illustrates that CTLA-4-Ig can induce colonic indoleamine 2,3-dioxygenase protein expression.

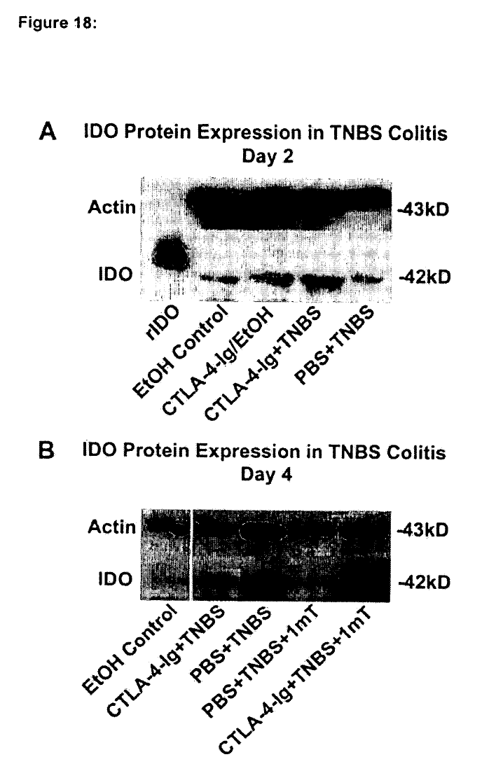

As illustrated in FIG. 18, increased IDO protein expression can be detected in the colons of mice receiving intra-peritoneal CTLA-4-Ig. In the setting of TNBS colitis, induction of IDO protein typically occurs around Days 3 to 4 (Gurtner et al., Gastroenterology 125, 1762-1773, 2003). As shown in FIG. 18A, at Day 2, IDO expression was distinctly greater in EtOH enema-treated animals that received CTLA-4-Ig instead of PBS. Comparable increases in colonic IDO protein expression could be detected in both EtOH treated controls and TNBS treated animals in response to CTLA-4-Ig administration. PBS treated animals receiving either EtOH enemas or TNBS had baseline IDO expression at Day 2. As shown in FIG. 18B, on Day 4, there was increased IDO expression in CTLA-4-Ig and TNBS treated animals relative to control animals. However there was essentially basal IDO protein expression in TNBS+PBS+1-mT treated animals. Animals receiving PBS+TNBS and CTLA-4-Ig+TNBS+1-mT had comparable marked increases in IDO protein expression compared to both control animals as well as TNBS+CTLA-4-Ig treated animals. Animals receiving TNBS+PBS+1-mT had a diminished IDO protein expression comparable to EtOH+PBS treated control animals. This figure is representative of three separate experiments. These data indicate that CTLA-4-Ig can stimulate or enhance colonic IDO expression, even in the presence of an IDO inhibitor.

### EXAMPLE 18

This example illustrates that CTLA-4-Ig inhibits Colonic TNFα mRNA expression but does not affect IL12 mRNA expression in the setting of TNBS colitis.

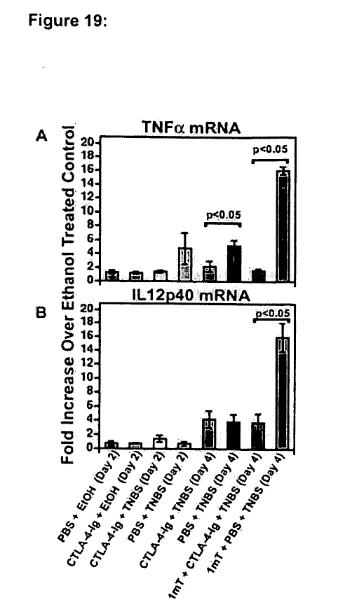

The data illustrated in FIG. 19A indicate that CTLA-4-Ig administration essentially had no effect on TNFα mRNA expression in EtOH treated control animals. TNFα mRNA expression was essentially baseline (<1.5 fold induction) in the CTLA-4-Ig+TNBS treated mice at Day 2, while there was a 5 fold induction in the PBS+TNBS treated animals. A 5 fold induction was also seen at Day 4 of TNBS colitis in the PBS treated animals while there was a minimal 2 fold induction in the CTLA-4-Ig treated animals (p<0.05). In the setting of IDO inhibition with 1-mT in TNBS treated animals at Day 4, TNFα remained low at a 1.5 fold induction in CTLA-4-Ig treated animals while there was a marked 16 fold induction in the PBS treated animals.

The data illustrated in FIG. 19B indicate that CTLA-4-Ig had a negligible effect on IL12 expression in ETOH treated control animals. IL12 expression was essentially baseline in CTLA-4-Ig and PBS treated mice also receiving TNBS at Day 2. There was a consistent 4 fold induction of IL12 mRNA in TNBS treated animals at Day 4 regardless of CTLA-4-Ig treatment. Only IDO inhibition in the absence of CTLA-4-Ig administration was able to upregulate IL12 mRNA expression by 16 fold (p<0.05). These results indicate that CTLA-4-Ig can inhibit colonic TNFα mRNA expression but not IL12 mRNA expression in the setting of an animal model of inflammatory bowel disease.

### EXAMPLE 19

This example illustrates that CTLA-4-Ig induces expression of TGFβ1 mRNA but not Forkhead box P3 (Foxp3) mRNA.

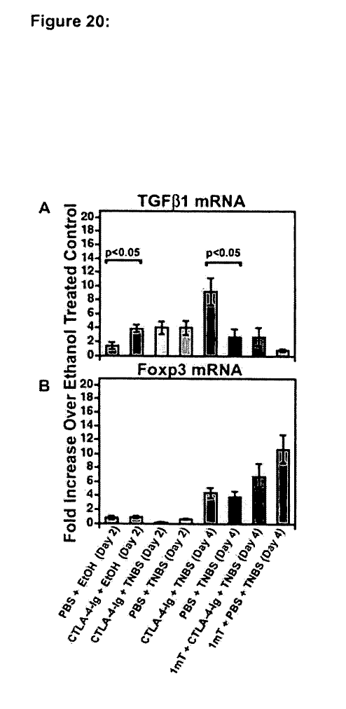

Mechanisms involving TGFβ1 upregulation have been associated with the abrogation of TNBS colitis (Neurath et al., Journal of Experimental Medicine 183, 2605-16 (1996); Kitani et al., Journal of Experimental Medicine 192, 41-52, 2000). In order to determine if an increase in TGFβ1 expression resulting from CTLA-4-Ig administration can be due to a proliferation or influx of CD4⁺CD25⁺ regulatory T cells that are associated with immune tolerance, we assessed Forkhead box P3 (Foxp3) mRNA expression as a specific marker the cells, as well as TGFβ1 mRNA expression. As shown in FIG. 20A, TGFβ1 mRNA could be induced by CTLA4-Ig in control animals, similar to induction of IDO and IFN-γ. In these experiments, TGFβ1 was induced 4 fold by CTLA-4-Ig administration alone (p<0.05). In ethanol treated control animals, TGFβ1 induction by CTLA-4-Ig was not altered by 1-mT administration (not shown). At Day 2 of TNBS colitis, TGFβ1 expression was comparable between CTLA-4-Ig and PBS treatment groups at about 4 fold induced. However at Day 4, TGFβ1 was induced 9 fold in CTLA-4-Ig and in TNBS treated animals. Animals receiving TNBS+PBS or CTLA-4-Ig treated animals also receiving TNBS+1-mT had significant reductions in TGβ1 expression and were comparably less than 3 fold induced (p<0.05). Animals receiving TNBS+1-mT but not receiving CTLA-4-Ig had even less TGFβ1 expression at 0.8 fold induced.

To determine if this induction of TGFβ1 was due to the presence of CD4⁺CD25⁺ regulatory T cells, we quantified Foxp3 mRNA as a specific marker for these cells (Khattri, Nature Immunology 4, 337-342, 2003). As shown in FIG. 20B, Foxp3 mRNA was not induced by CTLA-4-Ig in control animals. At Day 2 of TNBS colitis, there was a comparable suppression of Foxp3 mRNA in both CTLA-4-Ig and PBS treated animals (0.2 and 0.6 fold respectively). By Day 4 of TNBS colitis, there was a comparable induction of Foxp3 in both CTLA-4-Ig and PBS treated animals at about 4 fold. At Day 4, there was further induction of Foxp3 mRNA in both CTLA-4-Ig and PBS treated animals also receiving 1-mT at 7 fold and 11 fold respectively. These data indicate that CTLA-4-Ig administration can induce expression of TGβ1 mRNA but not Forkhead box P3 mRNA, in an animal model of inflammatory bowel disease. We conclude that induction of TGFβ1 in this animal model was not attributable to proliferation or influx of CD4⁺CD25⁺ regulatory T cells that are associated with immune tolerance.

It is to be understood that the embodiments of the present invention as set forth are not intended as being exhaustive or limiting of the invention, and that many alternatives, modifications, and variations will be apparent to those of ordinary skill in the art in light of the foregoing examples and detailed description. Accordingly, the embodiments set forth herein are intended to embrace all such alternatives, modifications, and variations that fall within the spirit and scope of the following claims.

All references cited in this specification are hereby incorporated by reference in their entireties. Any discussion of references cited herein is intended merely to summarize the assertions made by their authors and no admission is made that any reference or portion thereof constitutes relevant prior art. Applicants reserve the right to challenge the accuracy and pertinency of the cited references.

1. A method of treating inflammatory bowel disease in a patient, the method comprising administering to a patient in need thereof an immune tolerance-promoting amount of a ligand of B7 antigen comprised by antigen presenting cells of the patient's gastrointestinal tract.

2. A method of claim 1, wherein the ligand of B7 antigen provides a costimulatory blockade in the antigen presenting cells of the patient's gastrointestinal tract.

3. A method of claim 1, wherein the ligand of B7 antigen induces increased expression of indoleamine 2,3-dioxygenase in the antigen presenting cells of the patient's gastrointestinal tract.

4. A method of claim 1, wherein the ligand of B7 antigen comprises a cytotoxic T lymphocyte-associated antigen 4.

5. A method of claim 4, wherein the cytotoxic T lymphocyte-associated antigen 4 is selected from the group consisting of a cytotoxic T lymphocyte-associated antigen 4-Ig fusion polypeptide, a pegylated cytotoxic T lymphocyte-associated antigen 4-Ig fusion polypeptide and a combination thereof.

6. A method of claim 1, wherein the antigen presenting cells are professional antigen presenting cells.

7. A method of claim 1, wherein the antigen presenting cells are lamina propria mononuclear cells.

8. A method of claim 1, wherein the antigen presenting cells are macrophages or dendritic cells.

9. A method of claim 1, wherein the patient is a human patient.

10. A method of claim 1, wherein the inflammatory bowel disease is ulcerative colitis.

11. A method of claim 1, wherein the inflammatory bowel disease is Crohn's disease.

12. A method of claim 1, wherein the administering comprises administering systemically.

13. A method of claim 12, wherein the administering systemically comprises administering systemically by intravenous infusion.

14. A method of claim 1, further comprising administering at least one substance selected from the group consisting of 5-aminosalicylates, corticosteroids, azathioprine and infliximab.

15. A method of downregulating a T helper 1 cell proliferative response in inflammation within the gastrointestinal tract in a mammalian subject having inflammatory bowel disease, the method comprising administering to a subject in need thereof a pharmaceutical composition comprising an immune tolerance-promoting amount of a ligand of B7 antigen comprised by antigen presenting cells of the subject's gastrointestinal tract.

16. A method of claim 15, wherein the pharmaceutical composition comprising an immune tolerance-promoting amount of a ligand of B7 antigen comprises an inducer of indoleamine 2,3-dioxygenase.

17. A method of claim 16, wherein the inducer increases expression of indoleamine 2,3-dioxygenase in antigen presenting cells of the patient's gastrointestinal tract.

18. A method of claim 15, wherein the ligand of B7 antigen comprises a cytotoxic T lymphocyte-associated antigen 4.

19. A method of claim 18, wherein the cytotoxic T lymphocyte-associated antigen 4 is selected from the group consisting of a cytotoxic T lymphocyte-associated antigen 4-Ig fusion polypeptide, a pegylated cytotoxic T lymphocyte-associated antigen 4-Ig fusion polypeptide, and a combination thereof.

20. A method of claim 15, wherein the antigen presenting cells are professional antigen presenting cells.

21. A method of claim 15, wherein the antigen presenting cells are lamina propria mononuclear cells, macrophages, and dendritic cells.

22. A method of claim 16, wherein the antigen presenting cells are macrophages or dendritic cells.

23. A method of claim 15, wherein the mammalian subject is a human.

24. A method of claim 15, wherein the inflammatory bowel disease is ulcerative colitis.

25. A method of claim 15, wherein the inflammatory bowel disease is Crohn's disease.

26. A method of claim 15, wherein the administering comprises administering systemically.

27. A method of claim 26, wherein the administering systemically comprises administering systemically by infusion.

28. A method of claim 15, further comprising administering a substance selected from the group consisting of 5-aminosalicylates, corticosteroids, azathioprine and infliximab.

29. A packaged pharmaceutical comprising:
an anti-inflammatory amount of a ligand of B7 antigen comprised by antigen presenting cells of a patient's gastrointestinal tract, in a pharmaceutically acceptable formulation; and
instructions for using the ligand of B7 antigen for treating inflammatory bowel disease in a patient in need thereof.

30. A packaged pharmaceutical of claim 29, wherein the ligand of B7 antigen comprises a cytotoxic T lymphocyte-associated antigen 4.

31. A packaged pharmaceutical of claim 30, wherein the cytotoxic T lymphocyte-associated antigen 4 is selected from the group consisting of a cytotoxic T lymphocyte-associated antigen 4-Ig fusion polypeptide, a pegylated cytotoxic T lymphocyte-associated protein 4-Ig, and a combination thereof.

32. A packaged pharmaceutical of claim 29, wherein the patient is a human patient.

33. A packaged pharmaceutical of claim 29, wherein the inflammatory bowel disease is ulcerative colitis.

34. A packaged pharmaceutical of claim 29, wherein the inflammatory bowel disease is Crohn's disease.

35. A packaged pharmaceutical of claim 29, wherein the ligand of B7 antigen is in a formulation suitable for intraperitoneal infusion.

36. A packaged pharmaceutical of claim 29, wherein the ligand of B7 antigen is in a formulation suitable for systemic administration.

37. A packaged pharmaceutical of claim 29, wherein the ligand of B7 antigen is in a formulation suitable for intravenous infusion.

38. A packaged pharmaceutical of claim 29, further comprising a substance selected from the group consisting of a 5-aminosalicylate, a corticosteroid, an azathioprine and a combination thereof, in a pharmaceutically acceptable formulation.

39. A method of treating inflammatory bowel disease, the method comprising selecting an agent on the basis of the agent being effective in inducing indoleamine 2,3-dioxygenase in antigen presenting cells, effective in blockading costimulation of T cell activation, or effective in both inducing indoleamine 2,3-dioxygenase in antigen presenting cells and blockading costimulation of T cell activation, and administering an effective amount of the agent to a patient in need thereof.

40. A method of claim 39, wherein the agent is selected on the basis of the agent being effective in inducing indoleamine 2,3-dioxygenase in antigen presenting cells.

41. A method of claim 40, wherein the antigen presenting cells are selected from the group consisting of lamina propria mononuclear cells, macrophages, dendritic cells and a combination thereof.

42. A method of claim 40, wherein the agent is selected from the group consisting of a bacterial lipopolysaccharide, an interferon-γ, and a cytotoxic T lymphocyte-associated antigen 4.

43. A method of claim 42, wherein the cytotoxic T lymphocyte-associated antigen 4 is selected from the group consisting of a cytotoxic T lymphocyte-associated antigen 4-Ig fusion polypeptide, a pegylated cytotoxic T lymphocyte-associated protein 4-Ig and a combination thereof.

44. A method of claim 39, wherein the agent is selected on the basis of the agent being effective in blockading costimulation of T cell activation.

45. A method of claim 44, wherein the agent is a cytotoxic T lymphocyte-associated antigen 4.

46. A method of claim 45, wherein the cytotoxic T lymphocyte-associated antigen 4 is selected from the group consisting of a cytotoxic T lymphocyte-associated antigen 4-Ig fusion polypeptide, a pegylated cytotoxic T lymphocyte-associated protein 4-Ig and a combination thereof.

47. A method of claim 39, further comprising monitoring the patient for a response to the agent, wherein the response is indicative of therapeutic benefit.

48. A method of claim 47, wherein the monitoring the patient for a response comprises detecting an increase in indoleamine 2,3-dioxygenase expression in the antigen presenting cells of the patient's gastrointestinal tract.

49. A method of claim 48, wherein the detecting an increase in indoleamine 2,3-dioxygenase expression comprises detecting an increase in indoleamine 2,3-dioxygenase protein levels.

50. A method of claim 48, wherein the detecting an increase in indoleamine 2,3-dioxygenase expression comprises detecting an increase in indoleamine 2,3-dioxygenase mRNA levels.

51. A method of claim 47, wherein the monitoring the patient for a response comprises detecting a blockade of costimulation of T cell activation in the patient.
